(19) United States
(12) Patent Application Publication
(10) Pub. No.: US 2022/0273629 A1
(43) Pub. Date: Sep. 1, 2022

(54) METHODS OF TREATING DISORDERS ASSOCIATED WITH ELEVATED LEVELS OF ANTIBODIES THAT INTERACT WITH THE NMDA RECEPTOR

(71) Applicants: Aptinyx Inc., Evanston, IL (US); Northwestern University, Evanston, IL (US)

(72) Inventors: Joseph R. Moskal, Evanston, IL (US); Roger A. Kroes, Lake Zurich, IL (US); Torsten M. Madsen, Evanston, IL (US)

(21) Appl. No.: 17/631,021

(22) PCT Filed: Jul. 30, 2020
(86) PCT No.: PCT/US2020/044181

§ 371 Date: Jan. 28, 2022

Related U.S. Application Data
(60) Provisional application No. 62/881,472, filed on Aug. 1, 2019.

(51) Int. Cl. A61K 31/438; A61P 25/28

(52) U.S. Cl. CPC A61K 31/438; A61P 25/28

(57) ABSTRACT

Methods of treating a disorder associated with elevated NMDAR antibodies in a patient in need thereof are provided comprising, for example, administering to the patient an effective amount of a spiro-β-lactam compound.

### CROSS-REFERENCE TO RELATED APPLICATIONS

This application claims the benefit of the filing date of U.S. Provisional Application No. 62/881,472, filed Aug. 1, 2019. The entire content of this application is hereby incorporated by reference herein in its entirety.

### BACKGROUND

An N-methyl- D-aspartate (“NMDA”) receptor is a postsynaptic, ionotropic receptor that is responsive to, among other things, the excitatory amino acids glutamate and glycine and the synthetic compound NMDA. The NMDA receptor is critical for glutamatergic neurotransmission and synaptic plasticity and controls the flow of both divalent and monovalent ions into the postsynaptic neural cell through a receptor associated channel.

The NMDA receptor is believed to consist of several protein chains embedded in the postsynaptic membrane. The first two types of subunits discovered so far form a large extracellular region, which probably contains most of the allosteric binding sites, several transmembrane regions looped and folded so as to form a pore or channel, which is permeable to Ca++, and a carboxyl terminal region. The opening and closing of the channel is regulated by the binding of various ligands to domains of the protein residing on the extracellular surface. The binding of the ligands is thought to affect a conformational change in the overall structure of the protein which is ultimately reflected in the channel opening, partially opening, partially closing, or closing.

Anti-NMDAR encephalitis is a severe but often reversible autoimmune encephalitis characterized by the presence of antibodies against synaptic NMDAR. The disorder predominantly affects children and young adults, and is sometimes associated with tumors. Anti-NMDAR antibodies alter the structure and/or function of the corresponding

NMDAR receptor causing synaptic dysfunction, which may underlie the psychiatric and neurological manifestations of the disease. Current treatments based on immunomodulation inadequately alleviate the neuropsychiatric manifestations of the disorder and in several documented cases exacerbate these symptoms.

Thus, a need continues to exist in the art for the development of an appropriate treatment for anti-NMDAR encephalitis, which is as yet lacking.

### SUMMARY

The disclosure is directed in part to methods of treating a disorder associated with elevated NMDAR antibodies in a patient in need thereof, comprising administering to the patient a pharmaceutically effective amount of a spiro-β-lactam compound, such as a disclosed compound. For example, provided herein is a method of treating anti-NMDAR encephalitis in a patient in need thereof, comprising administering to the patient a pharmaceutically effective amount of a spiro-β-lactam compound, such as a disclosed compound.

Contemplated patients may also suffer from a germ-cell tumor, e.g., an ovarian or testicular teratoma. In some embodiments, a contemplated patient may also suffer from cancer and/or another autoimmune disease.

In an embodiment, a contemplated method may further comprise identifying the patient as having NMDAR IgA, IgM, and/or IgG isotype antibodies, e.g., can include identifying the patient as having NMDAR IgG isotype antibodies.

Contemplated disorders associated with elevated NMDAR antibodies can include immunotherapy-responsive dementia, such as unclassified dementia, progressive supranuclear palsy, corticobasal syndrome, frontotemporal dementia, Lewy body dementia, and/or primary progressive aphasia and/or may include psychiatric manifestations such as psychoses, mania, depression, confusion, etc. For example, a contemplated patient may suffer from progressive nonfluent aphasia. Other contemplated methods including administering a disclosed compound (e.g. a spiro-β-lactam compound) to a patient suffering from a disorder associated with elevated NMDAR antibodies where the disorder is immunotherapy-responsive neurodegenerative disorder without dementia, immunotherapy-responsive schizophrenia or Rasmussen's encephalitis

Methods described herein relate at least in part to the treatment of disorders related to autoimmune-induced glutamatergic receptor dysfunction by administering a disclosed compound, e.g., may relate to the use of NMDAR modulators for the treatment of autoimmune induced NMDAR encephalitis.

### BRIEF DESCRIPTION OF THE FIGURES

FIG. 1 shows the results of a β-lactamase assay, which evaluates compounds A, B, and C for their ability to restore NR2B surface expression levels in hNR1/PSD95/NR2B expressing HEK cells following a 45 min incubation with purified patient serum IgG antibodies.

FIG. 2 shows the results a β-lactamase assay, which evaluates compounds A, B, and C for their ability to restore NR2B surface expression levels in hNR1/PSD95/NR2B expressing HEK cells following incubation with ANRE patient CSF.

FIG. 3 shows the results of a β-lactamase assay, which evaluates compounds A, B, C, D, E, F, G, and H for their ability to restore NR2B surface expression levels in hNR1/PSD95/NR2B expressing HEK cells following a 45 min incubation with purified patient serum IgG antibodies.

FIG. 4 shows the results of a β-lactamase assay monitoring NR2B surface expression in hNR1/PSD95/NR2B-expressing HEK cells over a 24 hour time period following antibody incubation.

FIG. 5A depicts the experimental protocol used to test the effects of focal application of NR1 antibodies on synaptic transmission and long-term potentiation (LTP) of synaptic strength at Schaffer collateral-CA1 synapses in hippocampal slices in vitro.

FIG. 5B shows a normalized fEPSP Slope as a function of time and a summary bar graph from experiments assessing the anti-NR1 antibody-mediated effects on hippocampal slice LTP in the presence or absence of compound A.

FIG. 6A shows the results of a β-lactamase assay monitoring NMDAR 2B trafficking in wild-type and mutant R393A receptors in the presence of compound A and in the presence or absence of ANRE patient IgG serum.

FIG. 6B shows the results of a β-lactamase assay monitoring NMDAR 2B trafficking in wild-type and mutant R393A receptors in the presence of compound B and in the presence or absence of ANRE patient IgG serum.

FIG. 6C shows the results of a β-lactamase assay monitoring NMDAR 2B trafficking in wild-type and mutant R393A receptors in the presence of compound C and in the presence or absence of ANRE patient IgG.

### DETAILED DESCRIPTION

Described herein are methods of restoring NMDA receptor and/or NMDA receptor subtype surface expression in a disorder associated with NMDAR antibody production, said method comprising of administering an agent, .e.g., a spiro-β-lactam compound, which are NMDAR modulators, to said subject. This disclosure also provides methods for mitigating the severity of, lowering the incidence of or treating disorders associated with elevated NMDAR antibodies, said methods comprising of administering agents, which are NMDAR modulators to said subject. In some embodiments anti-NMDAR encephalitis may be treated.

### A. Definitions

The term “alkyl,” as used herein, refers to a saturated straight-chain or branched hydrocarbon, such as a straight-chain or branched group of 1-6, 1-4, or 1-3 carbon atoms, referred to herein as C₁-C₆ alkyl, C₁-C₄ alkyl, and C₁-C₃ alkyl, respectively. For example, “C₁-C₆ alkyl” refers to a straight-chain or branched saturated hydrocarbon containing 1-6 carbon atoms. Examples of a C₁-C₆ alkyl group include, but are not limited to, methyl, ethyl, propyl, butyl, pentyl, hexyl, isopropyl, isobutyl, sec-butyl, tent-butyl, isopentyl, and neopentyl. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 3-methyl-2-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, and hexyl.

The term “cyano,” as used herein, refers to the radical —CN.

The terms “halo” or “halogen,” as used herein, refer to fluoro (F), chloro (Cl), bromo (Br), and/or iodo (I).

The terms “hydroxy” and “hydroxyl,” as used herein, refer to the radical —OH.

The term “cycloalkyl,” as used herein, refers to a monocyclic saturated or partially unsaturated hydrocarbon ring (carbocyclic) system, for example, where each ring is either completely saturated or contains one or more units of unsaturation, but where no ring is aromatic. A cycloalkyl can have 3-6 or 4-6 carbon atoms in its ring system, referred to herein as C₃-C₆ cycloalkyl or C₄-C₆ cycloalkyl, respectively. Exemplary cycloalkyl groups include, but are not limited to, cyclohexyl, cyclohexenyl, cyclopentyl, cyclopentenyl, cyclobutyl, and cyclopropyl.

The terms “heteroaryl” as used herein refers to a monocyclic aromatic 4-6 membered ring system containing one or more heteroatoms, for example one to three heteroatoms, such as nitrogen, oxygen, and sulfur. Where possible, said heteroaryl ring may be linked to the adjacent radical though carbon or nitrogen. Examples of heteroaryl rings include but are not limited to furan, thiophene, pyrrole, thiazole, oxazole, isothiazole, isoxazole, imidazole, pyrazole, triazole, pyridine, and pyrimidine.

As used herein, “heterocyclic ring” refers to a non-aromatic cycloalkyl group that contains at least one ring heteroatom selected from O, S, Se, N, P, and Si (e.g., O, S, and N), and optionally contains one or more double or triple bonds. A heterocyclic ring can have 3 to 24 ring atoms, for example, 3 to 20 ring atoms (e.g., 3-14 membered heterocyclic ring), 3 to 8 ring atoms, 3 to 6 ring atoms, or 5 to 6 ring atoms. One or more N, P, S, or Se atoms (e.g., N or S) in a heterocyclic ring may be oxidized (e.g., morpholine N-oxide, thiomorpholine S-oxide, thiomorpholine S,S-dioxide). In some embodiments, nitrogen or phosphorus atoms of heterocyclic rings can bear a substituent, for example, a hydrogen atom, an alkyl group, or other substituents as described herein. Heterocyclic rings can also contain one or more oxo groups, such as oxopiperidyl, dioxopiperidyl (e.g., 2,6-dioxopiperidyl), oxooxazolidyl, dioxo-(1H,3H)-pyrimidyl, oxo-2(1H)-pyridyl, and the like. Examples of heterocyclic rings include, among others, morpholinyl, thiomorpholinyl, pyranyl, imidazolidinyl, imidazolinyl, oxazolidinyl, pyrazolidinyl, pyrazolinyl, pyrrolidinyl, pyrrolinyl, tetrahydrofuranyl, tetrahydrothiophenyl, piperidinyl, piperazinyl, and the like. In some embodiments, heterocyclic rings can be substituted as described herein.

The term “oxo,” as used herein, refers to the radical ═O (double bonded oxygen).

The term “amino acid,” as used herein, includes any one of the following alpha amino acids: isoleucine, alanine, leucine, asparagine, lysine, aspartate, methionine, cysteine, phenylalanine, glutamate, threonine, glutamine, tryptophan, glycine, valine, proline, arginine, serine, histidine, and tyrosine. An amino acid also can include other art-recognized amino acids such as beta amino acids.

The term “compound,” as used herein, refers to the compound itself and its pharmaceutically acceptable salts, hydrates, esters and N-oxides including its various stereoisomers and its isotopically-labelled forms, unless otherwise understood from the context of the description or expressly limited to one particular form of the compound, i.e., the compound itself, a specific stereoisomer and/or isotopically-labelled compound, or a pharmaceutically acceptable salt, a hydrate, an ester, or an N-oxide thereof It should be understood that a compound can refer to a pharmaceutically acceptable salt, or a hydrate, an ester or an N-oxide of a stereoisomer of the compound and/or an isotopically-labelled compound.

The term “moiety,” as used herein, refers to a portion of a compound or molecule.

The compounds of the disclosure can contain one or more chiral centers and/or double bonds and therefore, can exist as stereoisomers, such as geometric isomers, and enantiomers or diastereomers. The term “stereoisomers,” when used herein, consists of all geometric isomers, enantiomers and/or diastereomers of the compound. For example, when a compound is shown with specific chiral center(s), the compound depicted without such chirality at that and other chiral centers of the compound are within the scope of the present disclosure, i.e., the compound depicted in two-dimensions with “flat” or “straight” bonds rather than in three dimensions, for example, with solid or dashed wedge bonds. Stereospecific compounds may be designated by the symbols “R” or “S,” depending on the configuration of substituents around the stereogenic carbon atom. The present disclosure encompasses all the various stereoisomers of these compounds and mixtures thereof Mixtures of enantiomers or diastereomers can be designated “(±)” in nomenclature, but a skilled artisan will recognize that a structure can denote a chiral center implicitly. It is understood that graphical depictions of chemical structures, e.g., generic chemical structures, encompass all stereoisomeric forms of the specified compounds, unless indicated otherwise.

As discussed herein, the compounds of the present disclosure can have a plurality of chiral centers. Each chiral center can be independently R, S, or any mixture of R and S. For example, in some embodiments, a chiral center can have an R:S ratio of between about 100:0 and about 50:50 (“racemate”), between about 100:0 and about 75:25, between about 100:0 and about 85:15, between about 100:0 and about 90:10, between about 100:0 and about 95:5, between about 100:0 and about 98:2, between about 100:0 and about 99:1, between about 0:100 and 50:50, between about 0:100 and about 25:75, between about 0:100 and about 15:85, between about 0:100 and about 10:90, between about 0:100 and about 5:95, between about 0:100 and about 2:98, between about 0:100 and about 1:99, between about 75:25 and 25:75, or about 50:50. Formulations of the disclosed compounds comprising a greater ratio of one or more isomers (i.e., R and/or S) may possess enhanced therapeutic characteristic relative to racemic formulations of a disclosed compounds or mixture of compounds.

Individual enantiomers and diastereomers of compounds of the present disclosure can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, (3) direct separation of the mixture of optical enantiomers on chiral liquid chromatographic columns, or (4) kinetic resolution using stereoselective chemical or enzymatic reagents. Racemic mixtures also can be resolved into their component enantiomers by well-known methods, such as chiral-phase gas chromatography or crystallizing the compound in a chiral solvent. Stereoselective syntheses, a chemical or enzymatic reaction in which a single reactant forms an unequal mixture of stereoisomers during the creation of a new stereocenter or during the transformation of a pre-existing one, are well known in the art. Stereoselective syntheses encompass both enantio—and diastereoselective transformations. See, for example, Carreira and Kvaerno, Classics in Stereoselective Synthesis, Wiley-VCH: Weinheim, 2009.

Geometric isomers, resulting from the arrangement of substituents around a carbon-carbon double bond or arrangement of substituents around a cycloalkyl or heterocyclic ring, can also exist in the compounds of the present disclosure. The symbol [symbol] denotes a bond that may be a single, double or triple bond as described herein. Substituents around a carbon-carbon double bond are designated as being in the “Z” or “E” configuration, where the terms “Z” and “E” are used in accordance with IUPAC standards. Unless otherwise specified, structures depicting double bonds encompass both the “E” and “Z” isomers.

Substituents around a carbon-carbon double bond alternatively can be referred to as “cis” or “trans,” where “cis” represents substituents on the same side of the double bond and “trans” represents substituents on opposite sides of the double bond. The arrangement of substituents around a carbocyclic ring can also be designated as “cis” or “trans.” The term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

The disclosure also embraces isotopically-labeled compounds which are identical to those compounds recited herein, except that one or more atoms are replaced by an atom having an atomic mass or mass number different from the atomic mass or mass number usually found in nature. Examples of isotopes that can be incorporated into compounds described herein include isotopes of hydrogen, carbon, nitrogen, oxygen, phosphorus, fluorine and chlorine, such as ²H (“D”), ³H, ¹³C, ¹⁴C, ¹⁵N, ¹⁸O, ¹⁷O, ³¹P, ³²P, ³⁵S, ¹⁸F, and ³⁶Cl, respectively. For example, a compound described herein can have one or more H atoms replaced with deuterium.

Certain isotopically-labeled compounds (e.g., those labeled with ³H and ¹⁴C) can be useful in compound and/or substrate tissue distribution assays. Tritiated (i.e., ³H) and carbon-14 (i.e., ¹⁴C) isotopes can be particularly preferred for their ease of preparation and detectability. Further, substitution with heavier isotopes such as deuterium (i.e., ²H) can afford certain therapeutic advantages resulting from greater metabolic stability (e.g., increased in vivo half-life or reduced dosage requirements) and hence can be preferred in some circumstances. Isotopically-labeled compounds can generally be prepared by following procedures analogous to those disclosed herein, for example, in the Examples section, by substituting an isotopically-labeled reagent for a non-isotopically-labeled reagent.

The phrases “pharmaceutically acceptable” or “pharmacologically acceptable,” as used herein, refer to compounds, molecular entities, compositions, materials and/or dosage forms that do not produce an adverse, allergic or other untoward reaction when administered to an animal, or a human, as appropriate. For human administration, preparations should meet sterility, pyrogenicity, and general safety and purity standards as required by FDA Office of Biologics standards.

The phrases “pharmaceutically acceptable carrier” and “pharmaceutically acceptable excipient,” as used herein, refer to any and all solvents, dispersion media, coatings, isotonic and absorption delaying agents, and the like, that are compatible with pharmaceutical administration. Pharmaceutical acceptable carriers can include phosphate buffered saline solution, water, emulsions (e.g., such as an oil/water or water/oil emulsions), and various types of wetting agents. The compositions also can include stabilizers and preservatives.

The phrase “pharmaceutical composition,” as used herein, refers to a composition comprising at least one compound as disclosed herein formulated together with one or more pharmaceutically acceptable carriers. Pharmaceutical compositions can also contain other active compounds providing supplemental, additional, or enhanced therapeutic functions.

The terms “individual,” “patient,” and “subject,” as used herein, are used interchangeably and include any animal, including mammals, preferably mice, rats, other rodents, rabbits, dogs, cats, swine, cattle, sheep, horses, or primates, and more preferably, humans. The compounds described in the disclosure can be administered to a mammal, such as a human, but can also be administered to other mammals such as an animal in need of veterinary treatment, for example, domestic animals (e.g., dogs, cats, and the like), farm animals (e.g., cows, sheep, pigs, horses, and the like) and laboratory animals (e.g., rats, mice, guinea pigs, and the like). The mammal treated in the methods described in the disclosure is preferably a mammal in which treatment, for example, of pain or depression is desired.

The term “treating,” as used herein, includes any effect, for example, lessening, reducing, modulating, ameliorating, or eliminating, that results in the improvement of the condition, disease, disorder, and the like, including one or more symptoms thereof. Treating can be curing, improving, or at least partially ameliorating the disorder.

The term “disorder” refers to and is used interchangeably with, the terms “disease,” “condition,” or “illness,” unless otherwise indicated.

The term “modulation,” as used herein, refers to and includes antagonism (e.g., inhibition), agonism, partial antagonism, and/or partial agonism.

The phrases “pharmaceutically effective amount” and “therapeutically effective amount,” as used herein, refer to the amount of a compound (e.g., a disclosed compound) that will elicit the biological or medical response of a tissue, system, animal or human that is being sought by the researcher, veterinarian, medical doctor or other clinician. The compounds described in the disclosure can be administered in therapeutically effective amounts to treat a disease. A therapeutically effective amount of a compound can be the quantity required to achieve a desired therapeutic and/or prophylactic effect, such as an amount which results in lessening of a symptom of a disease or disorder such as anti-NMDAR encephalitis.

The phrase “pharmaceutically acceptable salt(s),” as used herein, refers to salt(s) of acidic or basic groups that can be present in compounds of the disclosure and/or used in the compositions of the disclosure. A pharmaceutically acceptable salt (e.g., acid or base) of a compound of the present disclosure, upon administration to a patient, is capable of providing a compound of this invention or an active metabolite or residue thereof.

The compounds disclosed herein can exist in a solvated form as well as an unsolvated form with pharmaceutically acceptable solvents such as water, ethanol, and the like, and it is intended that the disclosure embrace both solvated and unsolvated forms. In some embodiments, the compound is amorphous. In some embodiments, the compound is a single polymorph. In various embodiments, the compound is a mixture of polymorphs. In particular embodiments, the compound is in a crystalline form.

The term “prodrug,” as used herein, refers to compounds that are transformed in vivo to yield a disclosed compound or a pharmaceutically acceptable salt, hydrate or solvate of the compound. The transformation can occur by various mechanisms (such as by esterase, amidase, phosphatase, oxidative and or reductive metabolism) in various locations (such as in the intestinal lumen or upon transit into the intestine, blood or liver). Prodrugs are well known in the art (see, e.g., Rautio, Kumpulainen, et al., Nature Reviews Drug Discovery 2008, 7, 255). For example, if a compound described herein or a pharmaceutically acceptable salt, hydrate or solvate of the compound contains a carboxylic acid functional group, a prodrug can be an ester formed by the replacement of the hydrogen atom of the carboxylic acid group with a group such as (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as (β-dimethylaminoethyl), carbamoyl-(C₁-C₂)alkyl, N,N-di(C₁-C₂)alkylcarbamoyl-(C₁-C₂)alkyl, piperidino-(C₂-C₃)alkyl, pyrrolidino-(C₂-C₃)alkyl, or morpholino-(C₂-C₃)alkyl.

Similarly, if a compound described herein contains an alcohol functional group, a prodrug can be formed by the replacement of the hydrogen atom of the alcohol group with a group such as (C₁-C₆)alkanoyloxymethyl, 1-((C₁-C₆)alkanoyloxy)ethyl, 1-methyl-1((C₁-C₆)alkanoyloxy)ethyl (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanoyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate).

If a compound described herein incorporates an amine functional group, a prodrug can be formed, for example, by creation of an amide or carbamate, an N-acyloxyalkyl derivative, an (oxodioxolenyl) methyl derivative, an N-Mannich base, imine or enamine. In addition, a secondary amine can be metabolically cleaved to generate a bioactive primary amine, or a tertiary amine can metabolically cleaved to generate a bioactive primary or secondary amine. See, for example, Simplicio, et al., Molecules 2008, 13, 519 and references therein.

Unless defined otherwise, all technical and scientific terms used herein have the same meaning as commonly understood by one of ordinary skill in the art to which this disclosure pertains.

Throughout the description, where compositions and kits are described as having, including, or comprising specific components, or where processes and methods are described as having, including, or comprising specific steps, it is contemplated that, additionally, there are compositions and kits of the present disclosure that consist essentially of, or consist of, the recited components, and that there are processes and methods according to the present disclosure that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present disclosure, whether explicit or implicit herein. For example, where reference is made to a particular compound, that compound can be used in various embodiments of compositions of the present disclosure and/or in methods of the present disclosure, unless otherwise understood from the context. In other words, within this application, embodiments have been described and depicted in a way that enables a clear and concise application to be written and drawn, but it is intended and will be appreciated that embodiments can be variously combined or separated without parting from the present teachings and disclosure(s). For example, it will be appreciated that all features described and depicted herein can be applicable to all aspects of the disclosure(s) described and depicted herein.

The articles “a” and “an” are used in this disclosure to refer to one or more than one (i.e., to at least one) of the grammatical object of the article, unless the context is inappropriate. By way of example, “an element” means one element or more than one element.

The term “and/or” is used in this disclosure to mean either “and” or “or” unless indicated otherwise.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present disclosure also include the specific quantitative value itself, unless specifically stated otherwise. As used herein, the term “about” refers to a ±10% variation from the nominal value unless otherwise indicated or inferred.

Where a percentage is provided with respect to an amount of a component or material in a composition, the percentage should be understood to be a percentage based on weight, unless otherwise stated or understood from the context.

Where a molecular weight is provided and not an absolute value, for example, of a polymer, then the molecular weight should be understood to be an average molecule weight, unless otherwise stated or understood from the context.

It should be understood that the order of steps or order for performing certain actions is immaterial so long as the present disclosure remain operable. Moreover, two or more steps or actions can be conducted simultaneously.

At various places in the present specification, substituents are disclosed in groups or in ranges. It is specifically intended that the description include each and every individual subcombination of the members of such groups and ranges. For example, the term “C₁₋₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl. By way of other examples, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20. Additional examples include that the phrase “optionally substituted with 1-5 substituents” is specifically intended to individually disclose a chemical group that can include 0, 1, 2, 3, 4, 5, 0-5, 0-4, 0-3, 0-2, 0-1, 1-5, 1-4, 1-3, 1-2, 2-5, 2-4, 2-3, 3-5, 3-4, and 4-5 substituents.

The use of any and all examples, or exemplary language herein, for example, “such as” or “including,” is intended merely to illustrate better the present disclosure and does not pose a limitation on the scope of the disclosure unless claimed. No language in the specification should be construed as indicating any non-claimed element as essential to the practice of the present disclosure.

Further, if a variable is not accompanied by a definition, then the variable is defined as found elsewhere in the disclosure unless understood to be different from the context. In addition, the definition of each variable and/or substituent, for example, C₁-C₆ alkyl, R², R2, R^{b}), w and the like, when it occurs more than once in any structure or compound, can be independent of its definition elsewhere in the same structure or compound.

Definitions of the variables and/or substituents in formulae and/or compounds herein encompass multiple chemical groups. The present disclosure includes embodiments where, for example, i) the definition of a variable and/or substituent is a single chemical group selected from those chemical groups set forth herein, ii) the definition is a collection of two or more of the chemical groups selected from those set forth herein, and iii) the compound is defined by a combination of variables and/or substituents in which the variables and/or substituents are defined by (i) or (ii).

Various aspects of the disclosure are set forth herein under headings and/or in sections for clarity; however, it is understood that all aspects, embodiments, or features of the disclosure described in one particular section are not to be limited to that particular section but rather can apply to any aspect, embodiment, or feature of the present disclosure.

### B. Compounds

In some embodiments, a contemplated compound for use in a disclosed method is a spiro-β-lactam compound. In an embodiment, a contemplated compound may be represented, for example, by formula I or II:

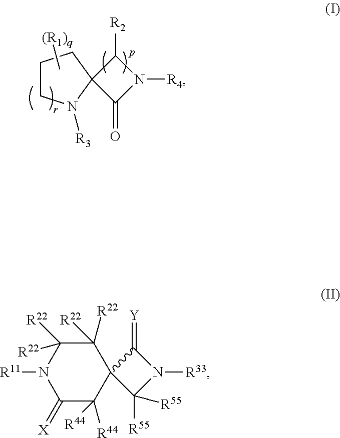

or a pharmaceutically acceptable salt, stereoisomer or N-oxide thereof.
wherein,
p is 1, 2, or 3;
q is 0, 1, 2 or 3;
r is 0, 1, 2, or 3;
   R₁ is selected, for each occurrence, from the group consisting of hydrogen, halogen, cyano, hydroxyl, C₁₋₆alkyl, phenyl, —C(O)—C₁₋₆alkyl, and —C(O)—O—C₁₋₆alkyl;
   R₂ is selected for each occurrence from the group consisting of hydrogen, halogen, cyano, hydroxyl, C₁₋₆alkyl, and phenyl;
   R₃ is selected from the group consisting of hydrogen, C₁₋₆alkyl, C(O)—C₁₋₆alkyl, S(O)_{w}—C₁₋₆alkyl (w is 0, 1 or 2), and C(O)—NH—C₁₋₆alkyl, wherein C₁₋₆alkyl is optionally substituted by one, two or three substituents each independently selected from the group consisting of OH, NR^{a}R^{b}, heteroaryl, phenyl, halogen, cyano, —C(O)—C₁₋₆alkyl, —C(O)—O—C₁₋₆alkyl, phenyl, and heteroaryl;
   R₄ is selected from the group consisting of: an amino acid, C₁₋₆alkyl, wherein C₁₋₆alkyl is optionally substituted by one, two or three substituents each independently selected from the group consisting of OH, NR^{a}R^{b}, C(O)NR^{a}R^{b}, C(O)—C₁₋₆alkyl, C(O)—O—C₁₋₆alkyl, phenyl, heteroaryl, or heterocycle), phenyl, heteroaryl, S(O)_{w}—C₁₋₆alkyl (w is 0, 1 or 2); R^{a} and R^{b} are each independently for each occurrence selected from the group consisting of hydrogen, —C₁-C₄alkyl, and —CH₂-phenyl; or R^{a} and R^{b} taken together with the nitrogen to which they are attached form a 4-7 membered heterocyclic ring;
   R¹¹ is selected from the group consisting of hydrogen, —C₁-C₆alkyl, —C(O)—C₁-C₆alkyl, —C(O)—O—C₁-C₆alkyl, —C₁-C₆alkylene-C₁-C₆cycloalkyl, and phenyl;

R²² is independently selected for each occurrence from the group consisting of hydrogen, cyano, —C₁-C₆alkyl, and halogen;
R³³ is selected from the group consisting of hydrogen, —C₁-C₆alkyl, —C(O)—R³¹, —C(O)—O—R³², and phenyl; wherein R³¹ is selected from the group consisting of hydrogen, —C₁-C₆alkyl, —C₁-C₆haloalkyl, —C₃-C₆cycloalkyl, and phenyl; R³² is selected from the group consisting of hydrogen, —C₁-C₆alkyl, —C₁-C₆haloalkyl, —C₃-C₆cycloalkyl, and phenyl; wherein any aforementioned C₁-C₆alkyl, independently for each occurrence, is optionally substituted by one, two or three substituents each independently selected from —C(O)NR^{a}R^{b}, —NR^{a}R^{b}, hydroxyl, —SH, phenyl, —O—CH₂-phenyl, and halogen; and any aforementioned phenyl, independently for each occurrence, is optionally substituted by one, two or three substituents each independently selected from —C(O)NR^{a}R^{b}, —NR^{a}R^{b}, —C₁-C₃alkoxy, hydroxyl, and halogen;
R⁴⁴ is independently selected for each occurrence from the group consisting of hydrogen, halogen, hydroxyl, cyano, phenyl, —C₁-C₄alkyl, —C₂₋₄alkenyl, —C₁₋₄alkoxy, —C(O)NR^{a}R^{b}, —NR^{a}R^{b}, —N(R^{a})-phenyl, —N(R^{a})—C₁-C₆alkylene-phenyl, —N(R^{a})—C(O)—C₁-C₆alkyl, —N(R^{a})—C(O)—C₁-C₆alkylene-phenyl, —N(R^{a})—C(O)—O—C₁-C₆alkyl, and —N(R^{a})—C(O)—O—C₁-C₆alkylene-phenyl; wherein C₁-C₄alkyl, C₁-C₆alkylene, C₂-C₄alkenyl, C₁-C₄alkoxy, and phenyl are optionally substituted by one or more substituents selected from R^{P}; or two R⁴⁴ moieties, when present on adjacent carbons, form a 3-membered carbocyclic ring taken together with the adjacent carbons to which they are attached, optionally substituted by one or two substituents independently selected from the group consisting of halogen, hydroxyl, —C₁-C₃alkyl, —C₁-C₃alkoxy, —C(O)NR^{a}R^{b}, and —NR^{a}R^{b}; R^{a} and R^{b} are each independently for each occurrence selected from the group consisting of hydrogen, —C₁-C₄alkyl, and —CH₂-phenyl; or R^{a} and R^{b} taken together with the nitrogen to which they are attached form a 4-7 membered heterocyclic ring;
R⁵⁵ is independently selected for each occurrence from the group consisting of hydrogen, —C₁-C₃alkyl, phenyl, and halogen; wherein phenyl is optionally substituted by one or more substituents selected from R^{P}; or two R⁵⁵ moieties together with the carbon to which they are attached form a carbonyl moiety or thiocarbonyl moiety.

In some embodiments, a contemplated compound for use in a disclosed method is represented by formula I:

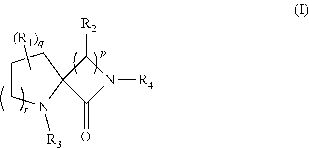

or a pharmaceutically acceptable salt, stereoisomer or N-oxide thereof.
wherein,
p is 1, 2, or 3;
q is 0, 1, 2 or 3;
r is 0, 1, 2, or 3.

In some embodiments R₁ is H.

In some embodiments R₂ is H.

In some embodiments R₃ is selected from a group consisting of hydrogen, C₁-C₆alkyl, C(O)—C₁₋₆alkyl, and S(O)_{w}—C₁₋₆alkyl (w is 0, 1 or 2).

In some embodiments R₃ is hydrogen or C(O)—C₁₋₆alkyl; wherein C₁₋₆alkyl is selected from a group consisting of, methyl, ethyl, and isopropyl.

In some embodiments R₃ is:

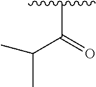 [SMILES: *C(=O)C(C)C]

In some embodiments R₄ is an amino acid and C₁₋₆alkyl; wherein C₁₋₆alkyl is optionally substituted by one, two or three substituents each independently selected from the group consisting of OH, NR^{a}R^{b}, —C(O)NR^{a}R^{b}, C(O)—C₁₋₆alkyl, —C(O)—O—C₁₋₆alkyl, phenyl, heteroaryl, and heterocycle; wherein, R^{a} and R^{b} are each independently selected for each occurrence from the group consisting of hydrogen and —C₁-C₆alkyl.

In some embodiments R₄ is:

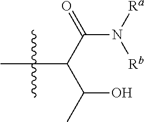

wherein R^{a} and R^{b} are each independently selected for each occurrence from the group consisting of hydrogen and —C₁-C₆alkyl.

In some embodiments R₄ is:

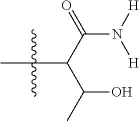 [SMILES: *C(C(N)=O)C(C)O]

In some embodiments, a contemplated compound for use in a disclosed method is:

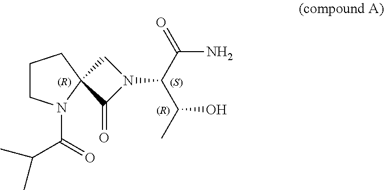

In some embodiments, a contemplated compound for use in a disclosed method is:

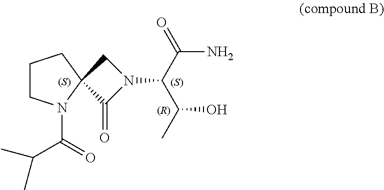

or a pharmaceutically acceptable salt, stereoisomer or N-oxide thereof.

In some embodiments, a contemplated compound for use in a disclosed method is selected from the group consisting of:

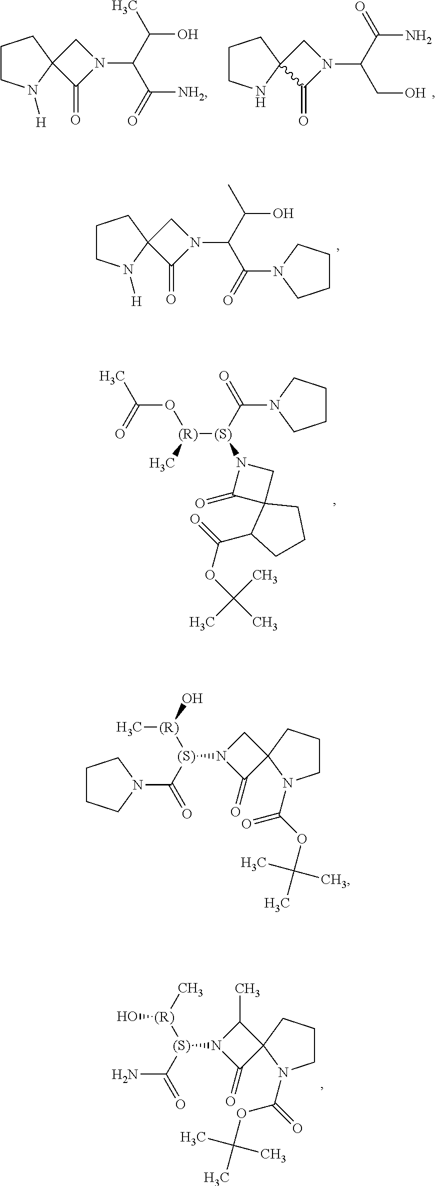

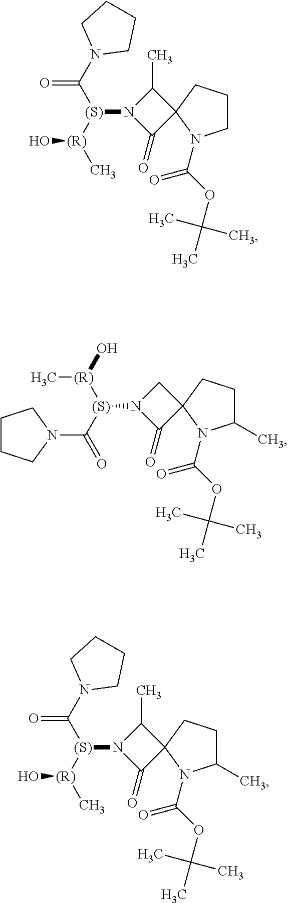

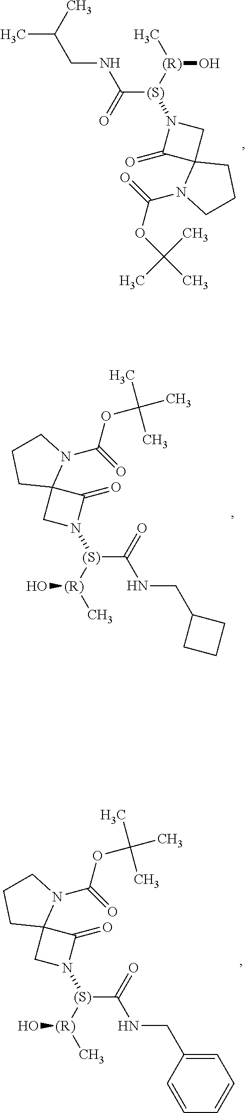

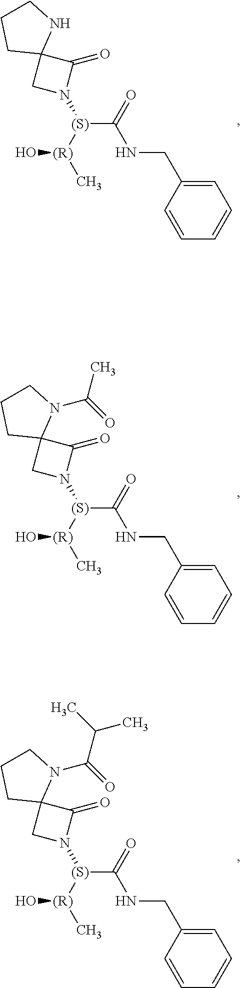

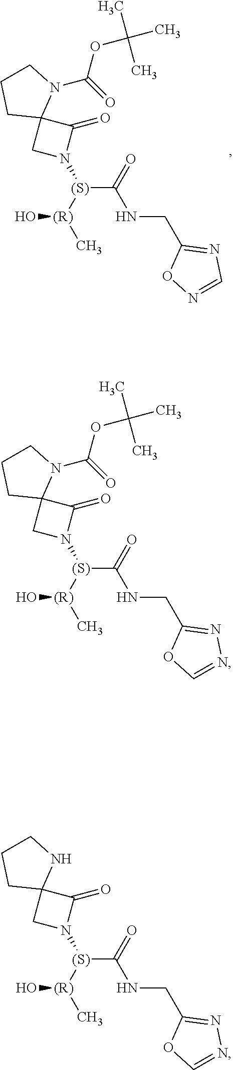

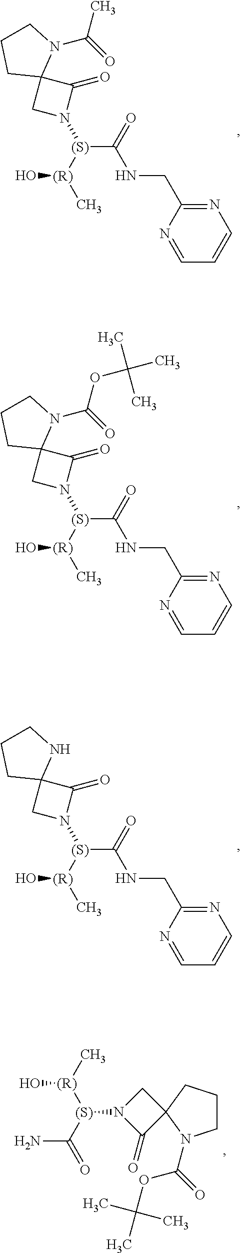

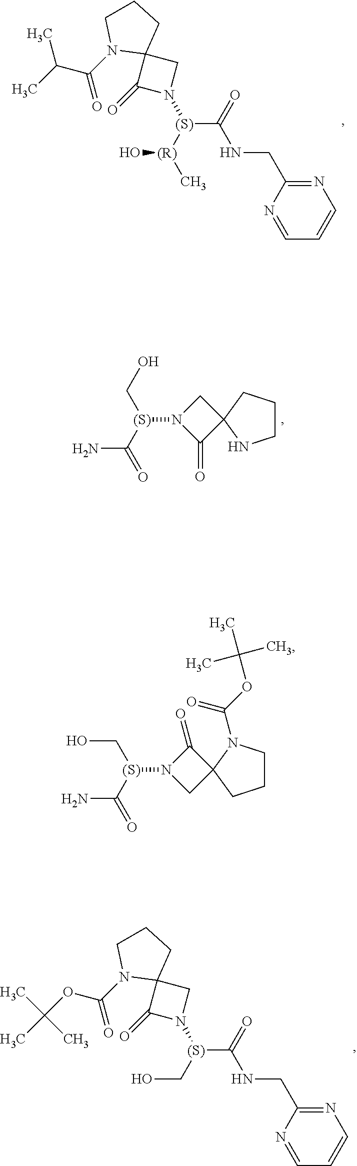

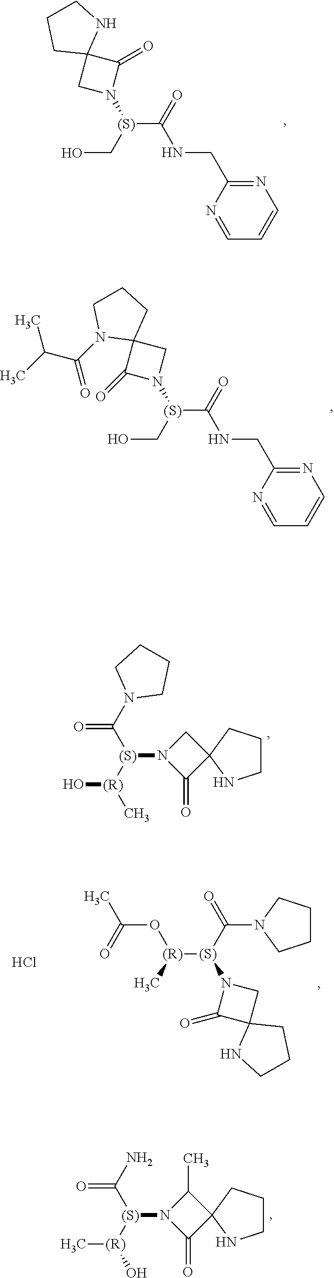

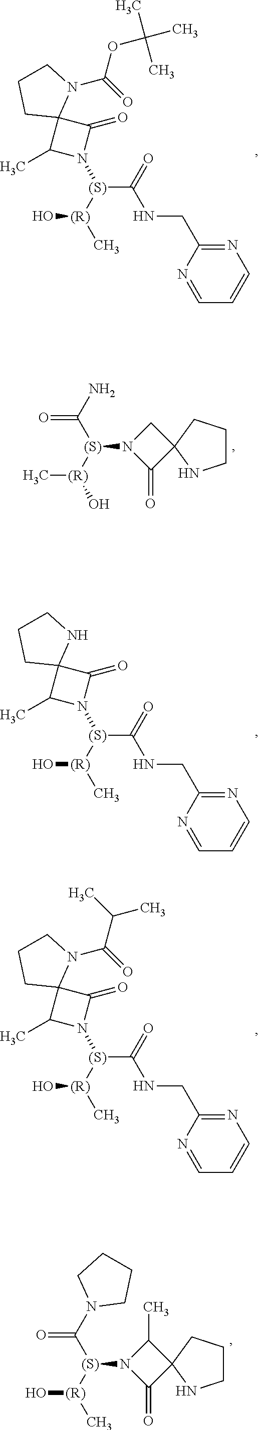

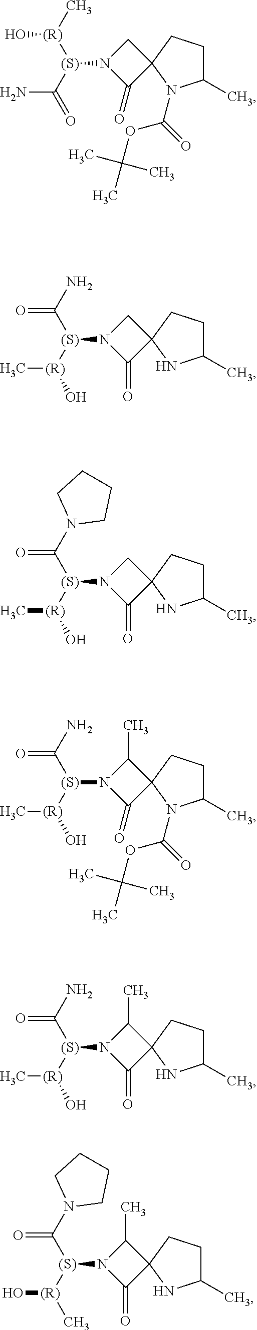

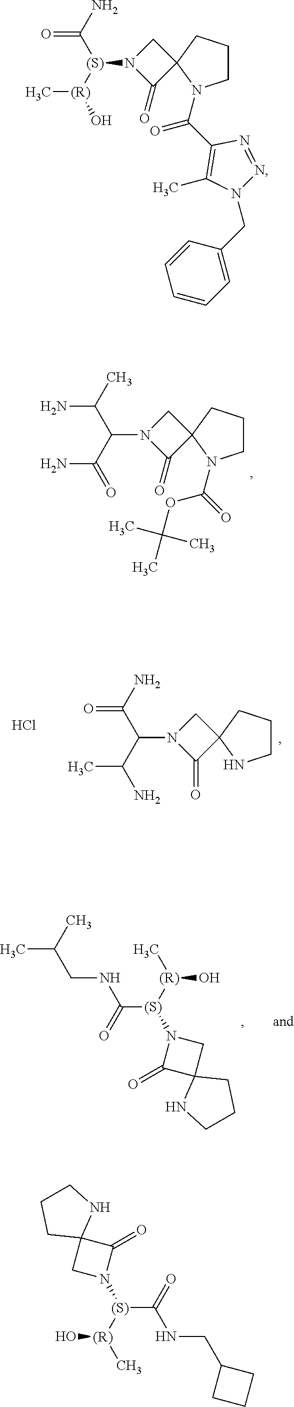

or a pharmaceutically acceptable salt, stereoisomer or N-oxide thereof.

In some embodiments, a contemplated compound for use in a disclosed method is represented by Formula (II):

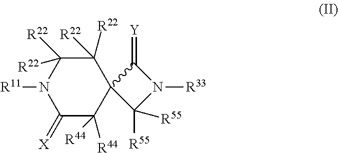

or a pharmaceutically acceptable salt, stereoisomer or N-oxide thereof.

In some embodiments, R¹¹ is hydrogen, and —C₁-C₆alkyl, wherein —C₁-C₆alkyl is optionally substituted by phenyl, where phenyl is optionally substituted by one, two or three substituents each independently selected from —C₁-C₃alkoxy and fluoro.

In some embodiments, R¹¹ is hydrogen.

In some embodiments, R²² is independently selected for each occurrence from the group consisting of hydrogen, and —C₁-C₆alkyl.

In some embodiments, R²² is hydrogen.

In some embodiments, R⁴⁴ is independently selected for each occurrence from the group consisting of hydrogen, halogen, hydroxyl, cyano, phenyl, —C₁-C₄alkyl, —C₂₋₄alkenyl, —C₁₋₄alkoxy, —C(O)NR^{a}R^{b}, —NR^{a}R^{b}; where R^{a} and R^{b} are each independently for each occurrence selected from the group consisting of hydrogen, —C₁-C₄alkyl, and —CH₂-phenyl.

In some embodiments, R⁴⁴ is hydrogen.

In some embodiments, R⁵⁵ is independently selected for each occurrence from the group consisting of hydrogen, —C₁-C₃alkyl, and halogen

In some embodiments, R⁵⁵ is hydrogen.

In some embodiments, R³³ is selected from the group consisting of hydrogen, and —C₁-C₆alkyl; where C₁-C₆alkyl is optionally substituted by one, two or three substituents each independently selected from hydroxyl, —SH, phenyl, —O—CH₂-phenyl, and halogen; and any aforementioned phenyl, independently for each occurrence, is optionally substituted by one, two or three substituents each independently selected from —C(O)NR^{a}R^{b}, —NR^{a}R^{b}, —C₁-C₃alkoxy, hydroxyl, and halogen.

In some embodiments, R³³ is:

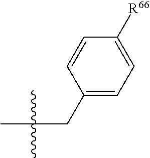

where:
R⁶⁶ is selected from the group consisting of hydrogen, halogen, —C₁-C₃alkoxy.

In some embodiments, R⁶⁶ is methoxy.

In some embodiments, the compound is:

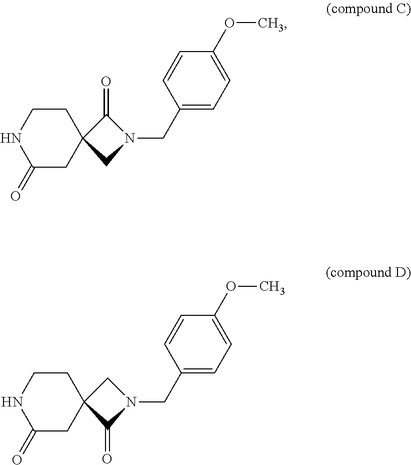

or a pharmaceutically acceptable salt, stereoisomer or N-oxide thereof.

In some embodiments, a contemplated compound for use in a disclosed method is selected from the group consisting of:

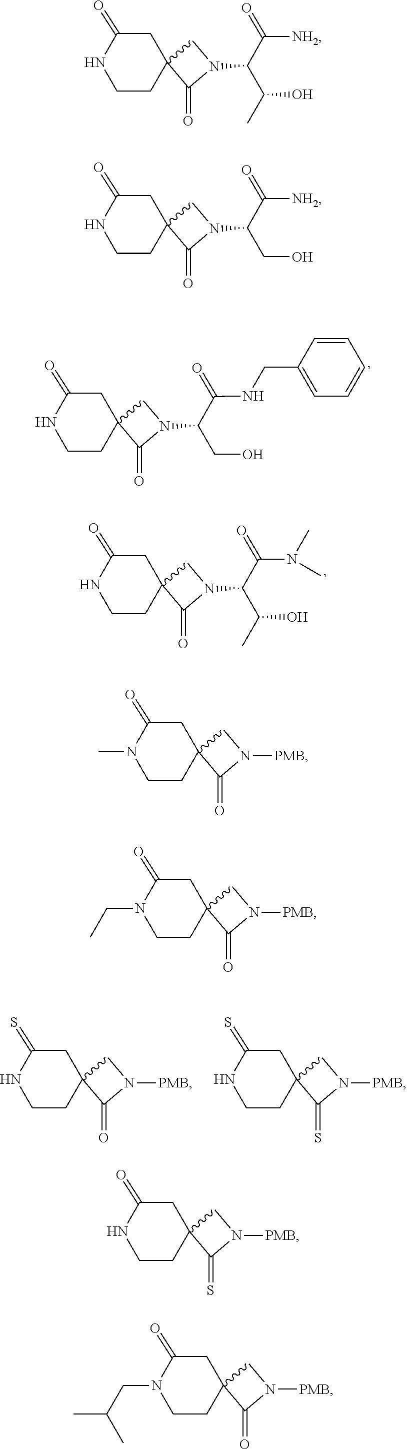

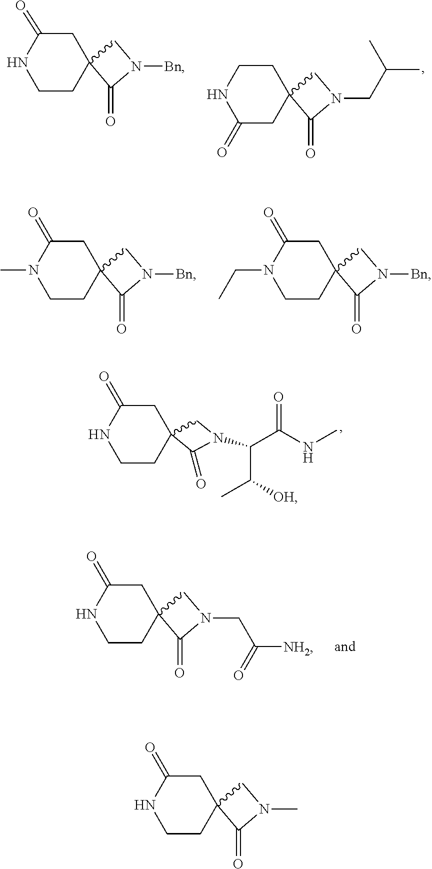

or a stereoisomer and/or pharmaceutically acceptable salt thereof.

In some embodiments, a contemplated compound for use in a disclosed method is represented by Formula (III):

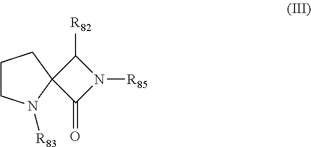

Or a pharmaceutically acceptable salt thereof, wherein
R₈₂ is H or —C₁-C₆ alkyl;
R₈₃ is selected from the group consisting of H, C₁-C₆ alkyl and a nitrogen protecting group;
R₈₅ is X, —C₁-C₆ alkyl-X and —C₁-C₆ alkylene-X, wherein X is selected from the group consisting of:
(i) phenyl;
(ii) heteroaryl including from 5 to 6 ring atoms wherein 1, 2, or 3 of the ring atoms are independently selected from the group consisting of N, NH, N(C₁-C₃ alkyl), O, and S; and
(iii) heterocyclyl including from 3 to 6 ring atoms wherein 1, 2, or 3 of the ring atoms are independently selected from the group consisting of N, NH, N(C1-C3 alkyl), O, and S; wherein
R₈₅ is optionally substituted with

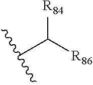

and
R₈₆ is selected from the group consisting of H, halogen, hydroxyl, cyano, —O—C(O)—C₁-C₆alkyl, C₁-C₆ alkyl, or C₁-C₆ alkoxy, and R₈₄ is H or C₁-C₆ alkyl.

In another embodiment, a contemplated compound for use in a disclosed method is selected from the group consisting of:

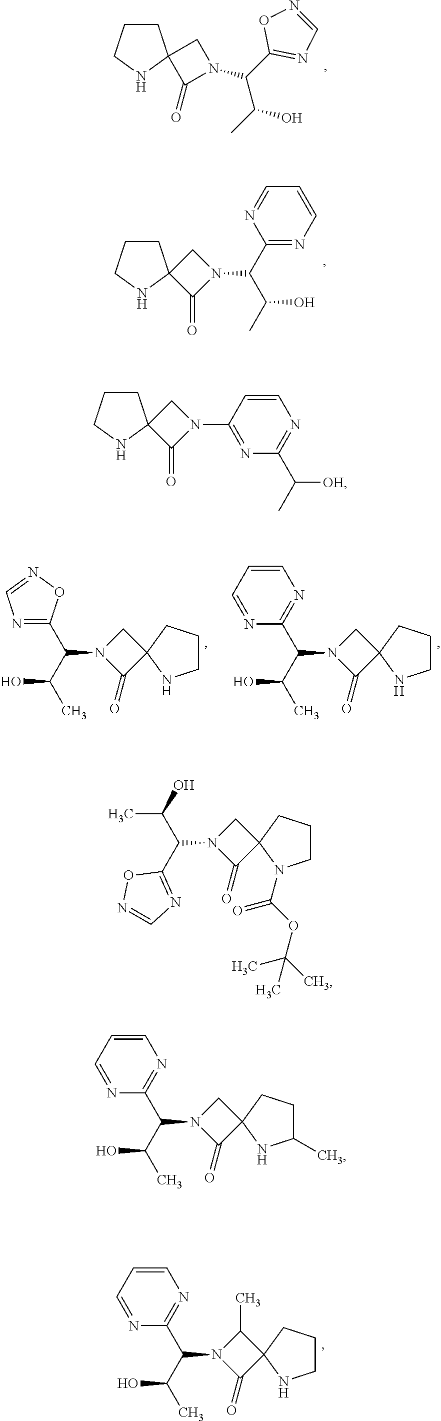

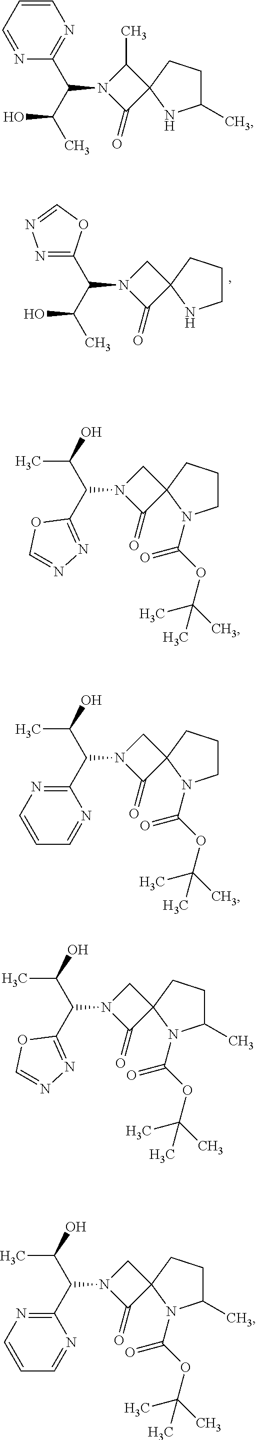

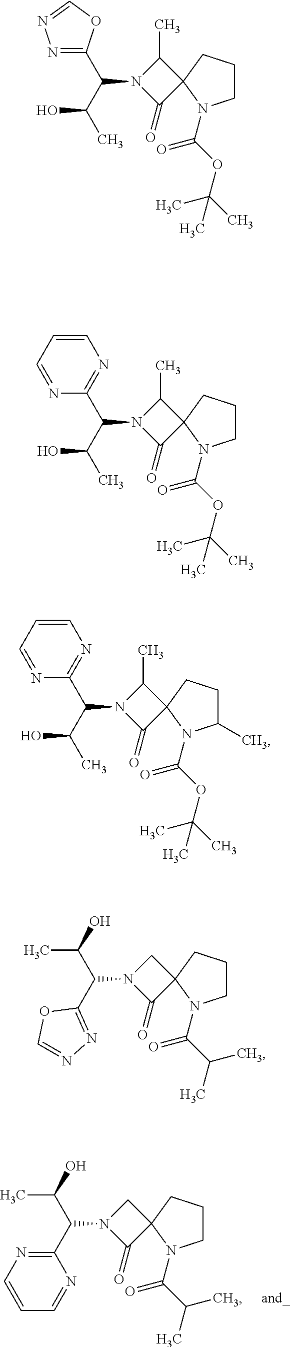

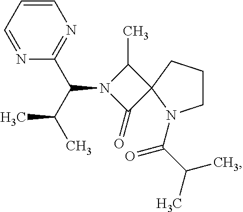

Or a stereoisomer and/or pharmaceutically acceptable salt thereof.

In some embodiments, a contemplated compound for use in a disclosed method is represented by formula (IV):

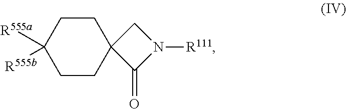

or a pharmaceutically acceptable salt and/or a stereoisomer thereof, wherein:
R¹¹¹ is selected from the group consisting of H, —C₁-C₄alkyl, —C₁-C₄alkyl-phenyl, —C(O)—R³¹, —C(O)—O—R³², —O—C₁-C₄alkyl-phenyl, phenyl, and —CH(R⁸⁸⁸)—C(O)—R⁹⁹⁹; wherein phenyl is optionally substituted by one, two or three substituents each independently selected from —C₁-C₄alkyl, —C₁-C₄alkoxy, hydroxyl, and halogen; R⁸⁸⁸ is selected from the group consisting of H and —C₁-C₄alkyl, wherein C₁-C₄alkyl is optionally substituted by one, two or three substituents each independently selected from —C(O)NR^{a}R^{b}, —NR^{a}—C(O)—C₁-C₄alkyl, —NR^{a}R^{b}, —SH, —C(O)—C₁-C₄alkyl, —C(O)—O—C₁-C₄alkyl, —O—C(O)—C₁-C₄alkyl, —C₁-C₄alkoxy, —COOH, hydroxyl, and halogen; R⁹⁹⁹ is selected from the group consisting of hydroxyl, —C₁-C₄alkoxy, and —NR^{a}R^{b};
R^{555a} is selected from the group consisting of H, hydroxyl, halogen, cyano, —C₁-C₄alkoxy, —O—C₁-C₄ alkyl-phenyl, —C₁-C₄alkyl, —C(O)—C₁-C₄alkyl, —NR^{a}—C(O)—C₁-C₄alkyl, —NR^{a}—C(O)—O—C₁-C₄alkyl, —NR^{a}R^{b}, and —NR^{a}CH(R¹⁰)—C(O)—R¹¹; wherein C₁-C₄alkyl is optionally substituted by one, two or three substituents each independently selected from —COOH, —C(O)NH₂, —NR^{a}R^{b}, —SH, —C(O)—C₁-C₄alkyl, —C(O)—O—C₁-C₄alkyl, —O—C(O)—C₁-C₄alkyl, —C₁-C₄alkoxy, phenyl, hydroxyl, and halogen; and phenyl, independently for each occurrence is optionally substituted by one, two or three substituents each independently selected from —C₁-C₄alkyl, —C₁-C₄haloalkyl, —C₁-C₄alkoxy, —NR^{a}R^{b}, hydroxyl, cyano, and halogen;
R^{555b} is selected from the group consisting of H, halogen, cyano, —C₁-C₄alkyl, and —C₁-C₄haloalkyl; or
or R^{555a} and R^{555b} taken together form an oxo group;

Contemplated compounds for use in a disclosed method may be, in another embodiment, selected from the group consisting of:

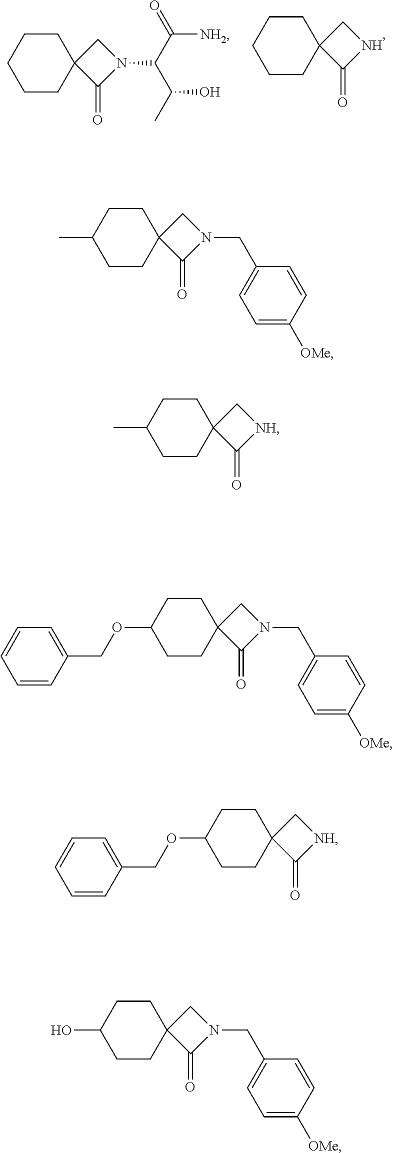

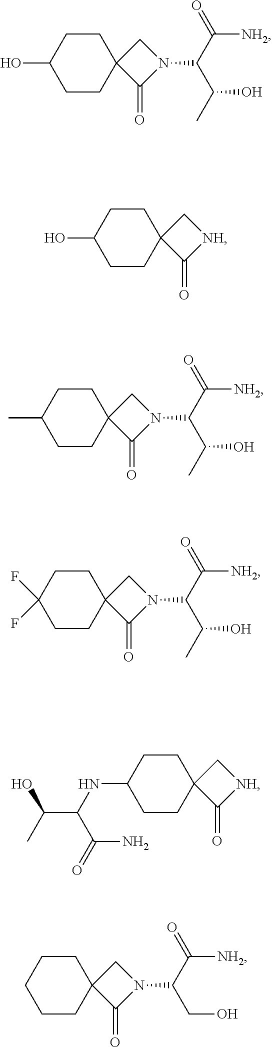

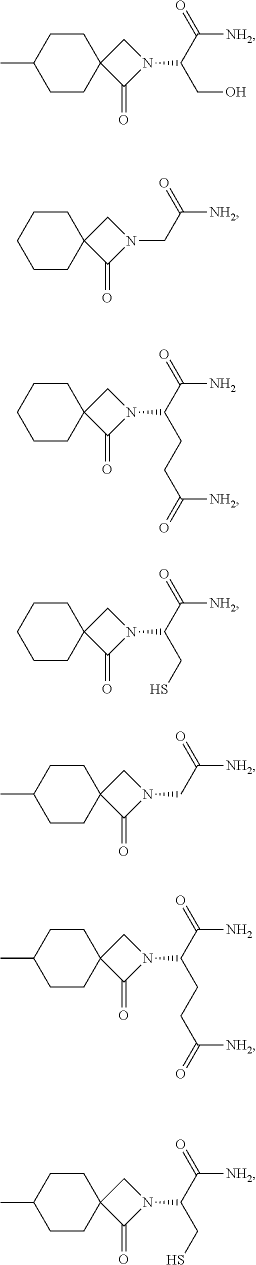

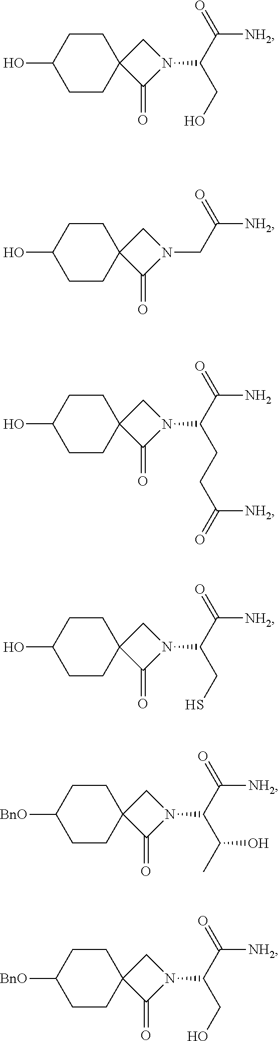

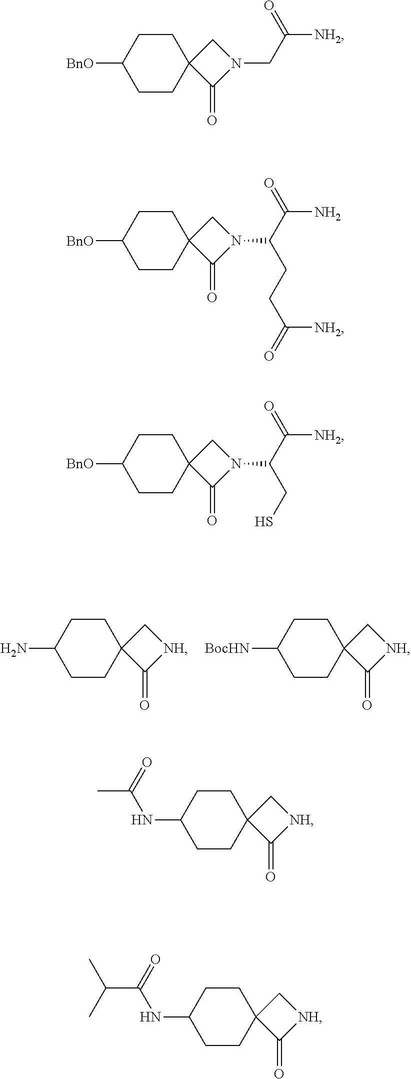

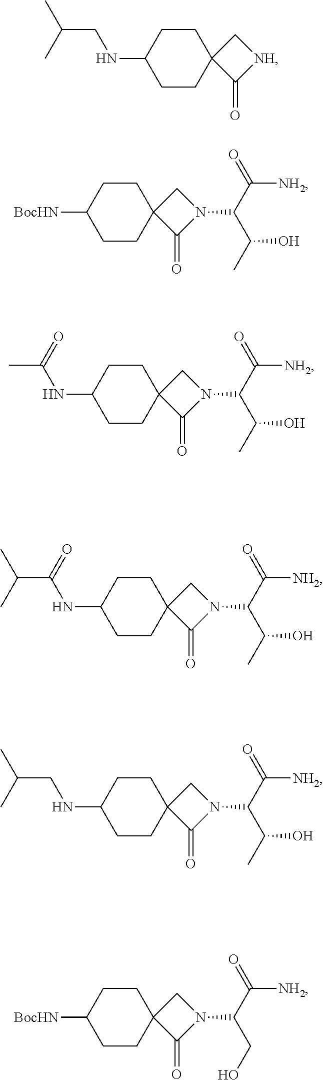

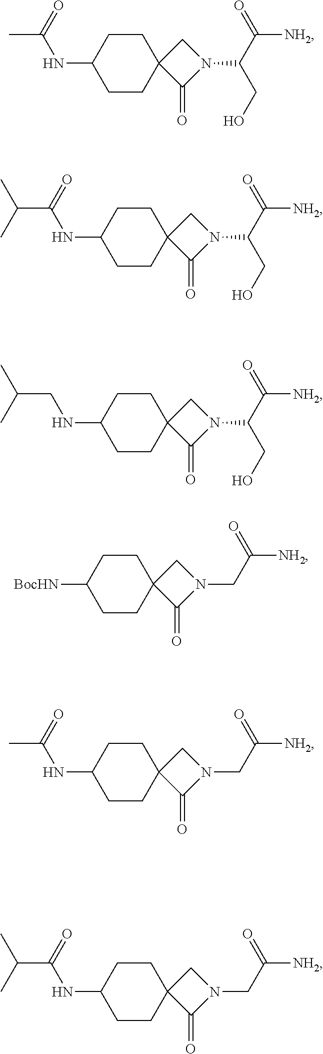

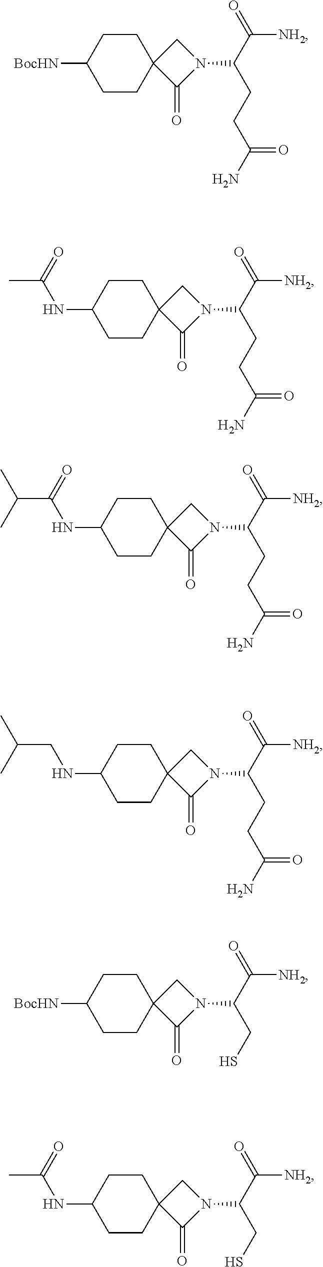

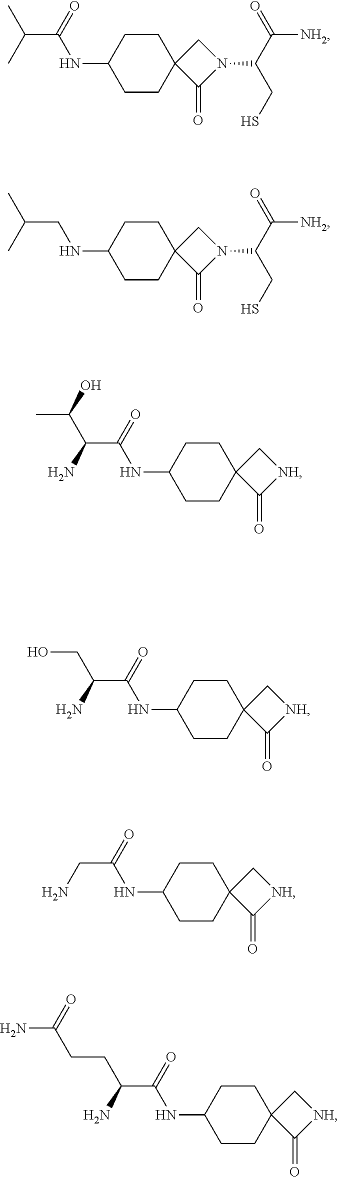

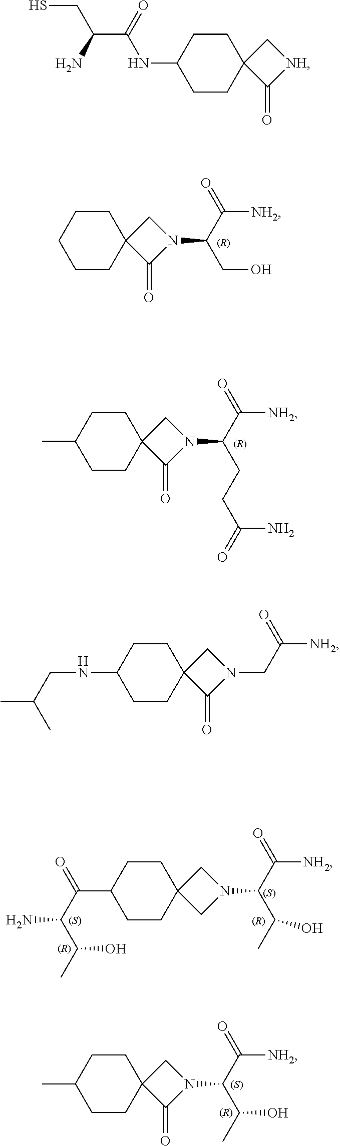

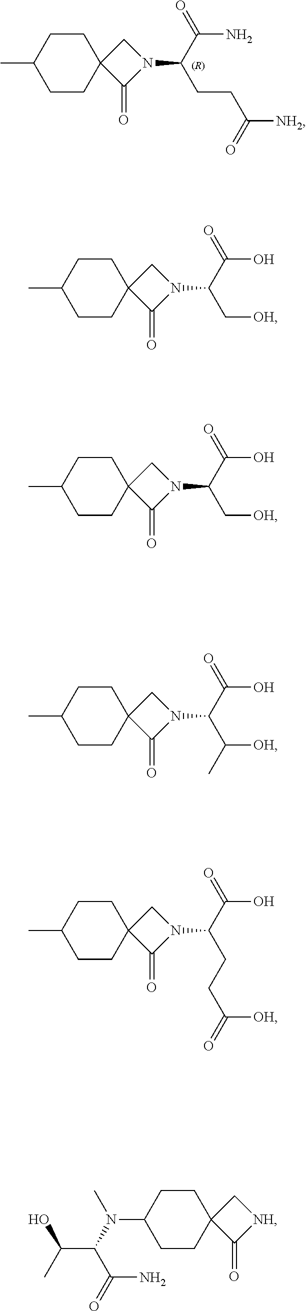

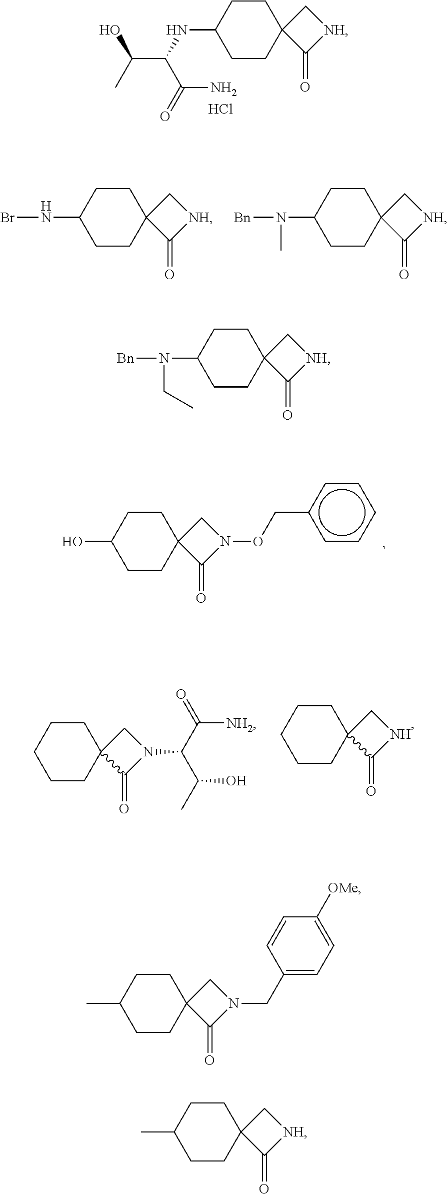

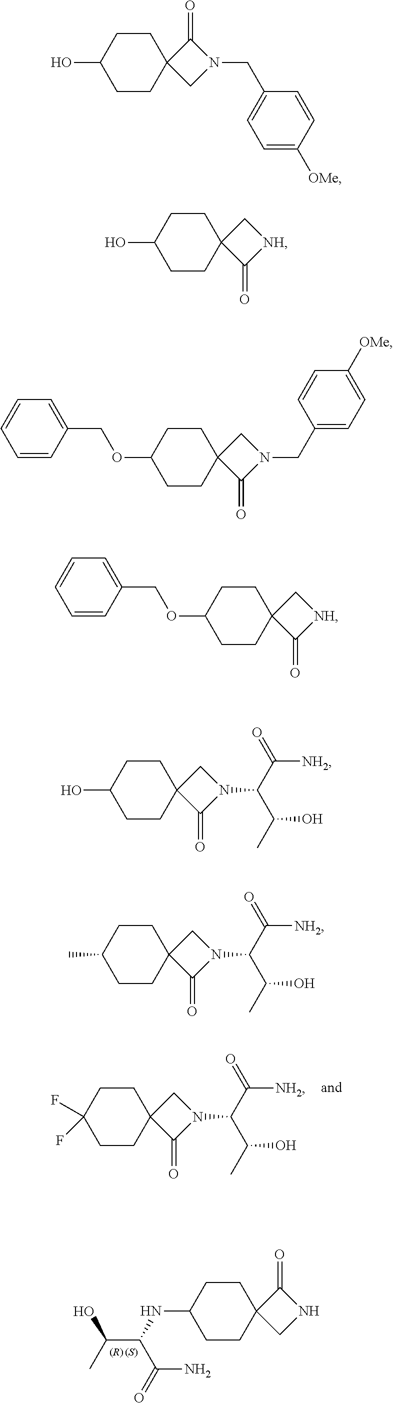

Or a stereoisomer and/or pharmaceutically acceptable salt thereof.

In some embodiments, a contemplated compound for use in a disclosed method is represented by Formula (V)

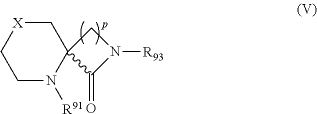

or a pharmaceutically acceptable salt and/or stereoisomer thereof, wherein:
X is O or NR⁹²;
p is 1 or 2;
R⁹¹ is selected from the group consisting of H, C₁-C₆alkyl, phenyl, —C(O)—C₁-C₆alkyl, and —C(O)—O—C₁-C₆ alkyl;
R⁹² is selected from the group consisting of H, C₁-C₆alkyl, phenyl, —C(O)—C₁-C₆alkyl, and —C(O)—O—C₁-C₆ alkyl;
R⁹³ is selected from the group consisting of H, C₁-C₆ alkyl, phenyl, —C(O)R³¹ and —C(O)OR³²; wherein R³¹ and R³² are each independently selected from the group consisting of H, C₁-C₆alkyl, —C₃-C₆cycloalkyl, and phenyl.

In some embodiments, a contemplated compound for use in a disclosed method is selected from the group consisting of:

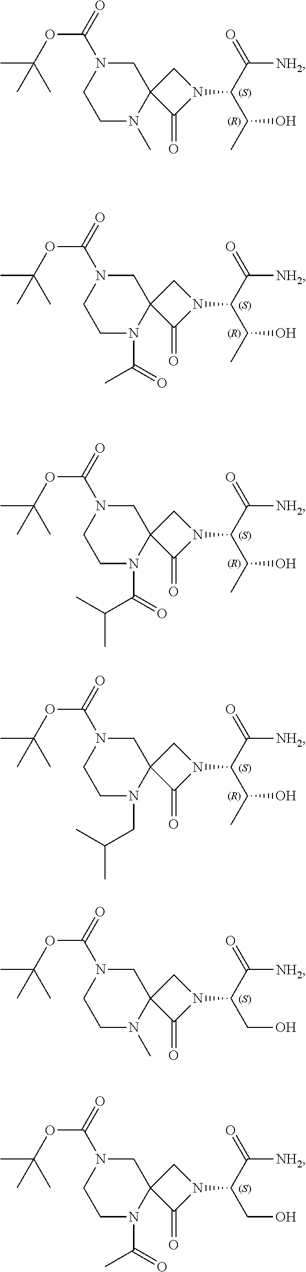

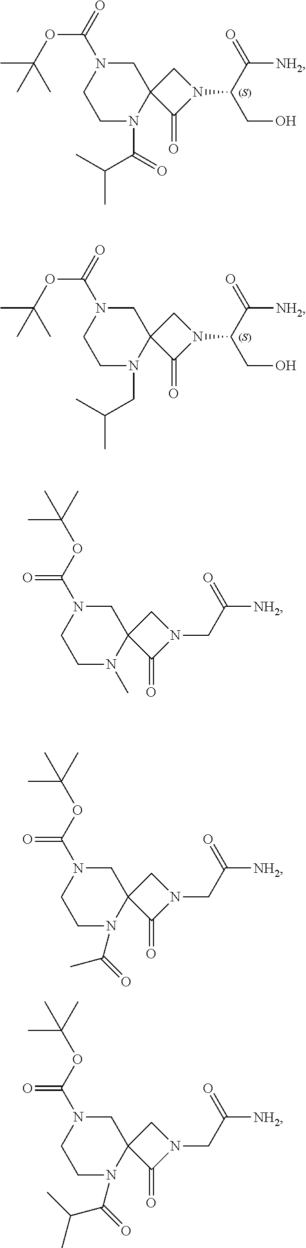

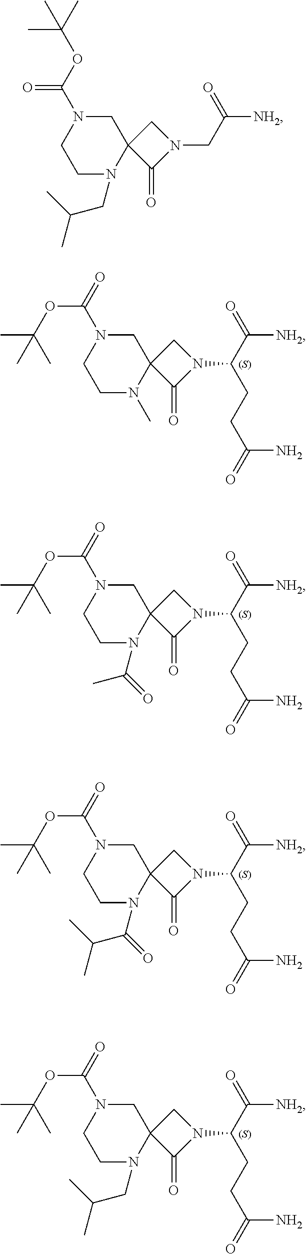

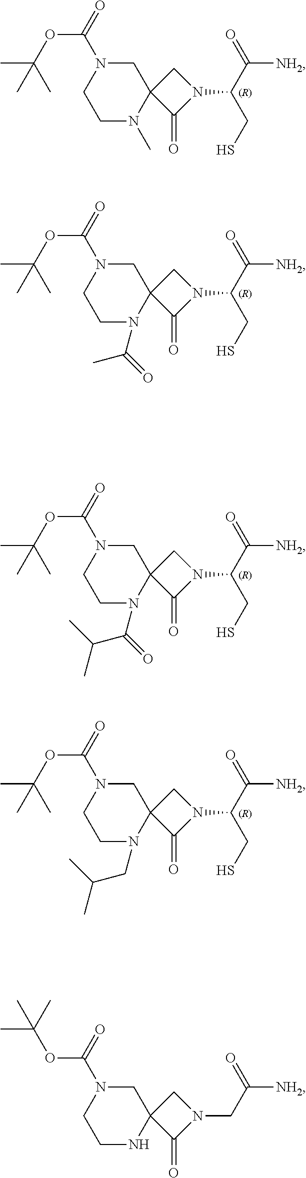

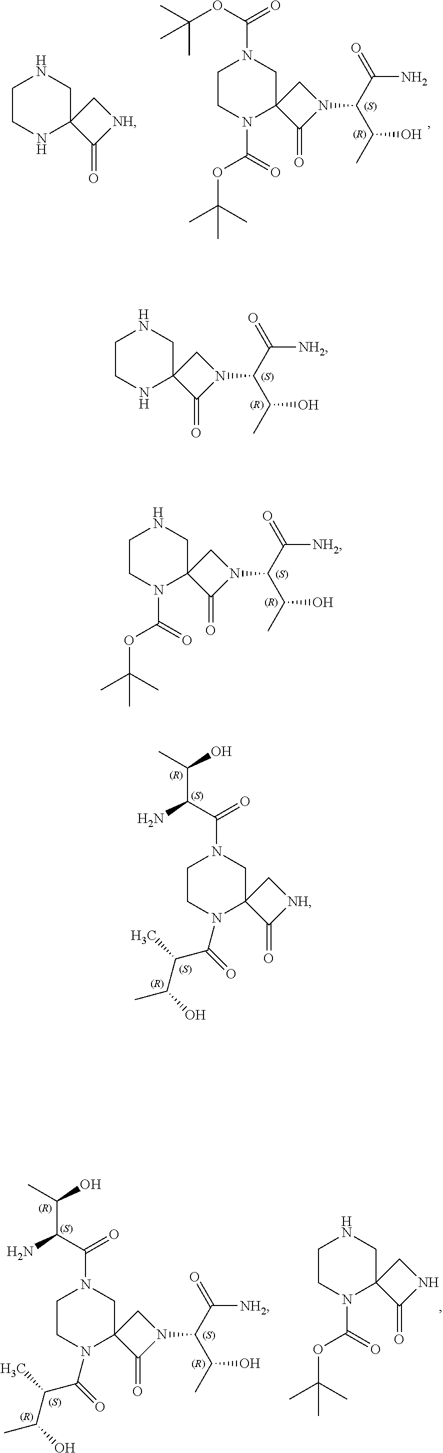

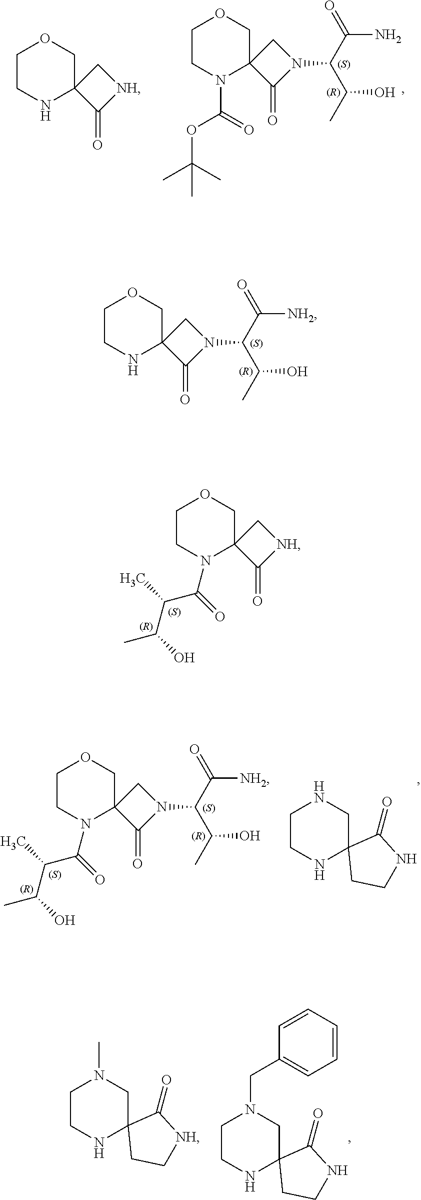

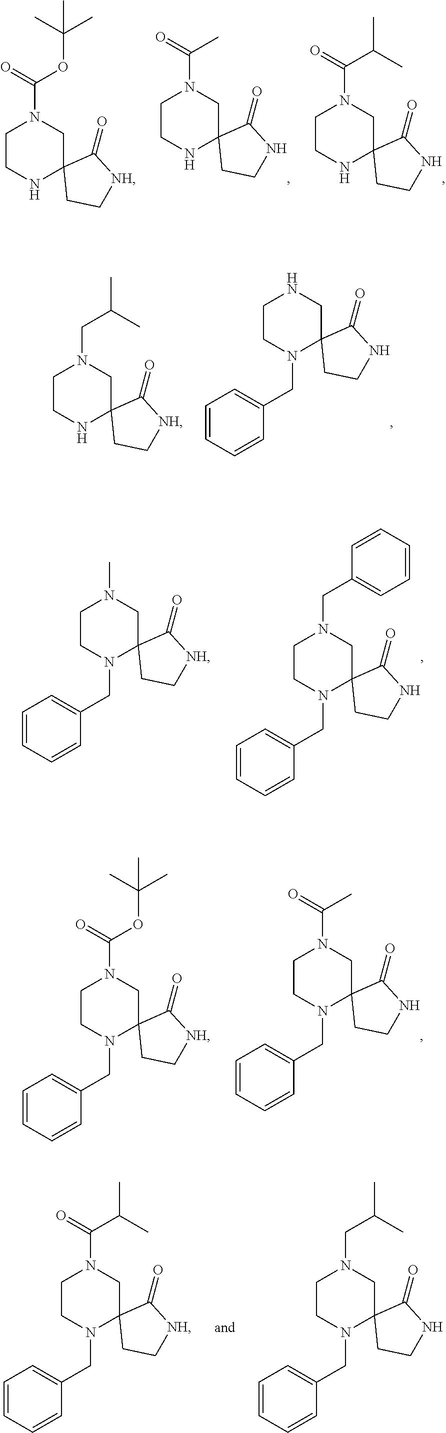

or a stereoisomer and/or pharmaceutically acceptable salt thereof.

In some embodiments, a contemplated compound for use in a disclosed method is represented by Formula (VI):

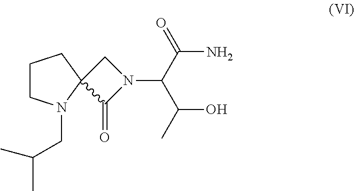 [SMILES: CC(C)CN1CCCC12CN(C(C(N)=O)C(C)O)C2=O]

or a stereoisomer and/or pharmaceutically acceptable salt thereof.

Contemplated compounds for use in a disclosed method may also be selected from the group consisting of:

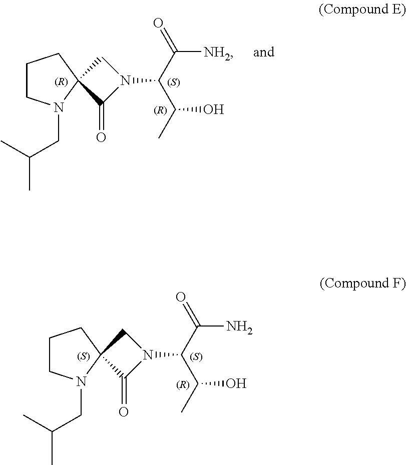

or a stereoisomer and/or pharmaceutically acceptable salt thereof.

In some embodiments, a contemplated compound for use in a disclosed method is represented by Formula (VII):

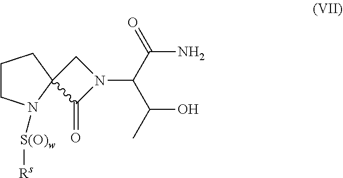

and stereoisomers and/or pharmaceutically acceptable salts thereof, wherein
R^{S} is C₁₋₃alkyl;
w is 0, 1 or 2;

Other contemplated compounds include:

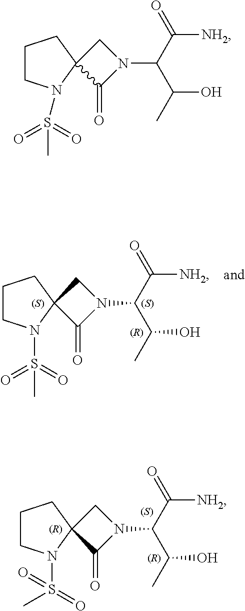

and stereoisomers and/or pharmaceutically acceptable salts thereof,

In some embodiments, a contemplated compound for use in a disclosed method is represented by Formula (VI):

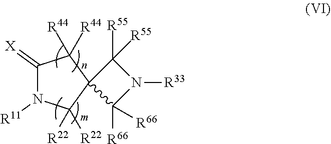

or a pharmaceutically acceptable salt, stereoisomer, and/or N-oxide thereof, wherein:
m is 0, 1 or 2;
n is 1 or 2;
X is O or S;

In some embodiments, a contemplated compound for use in a disclosed method is selected from the group consisting of:

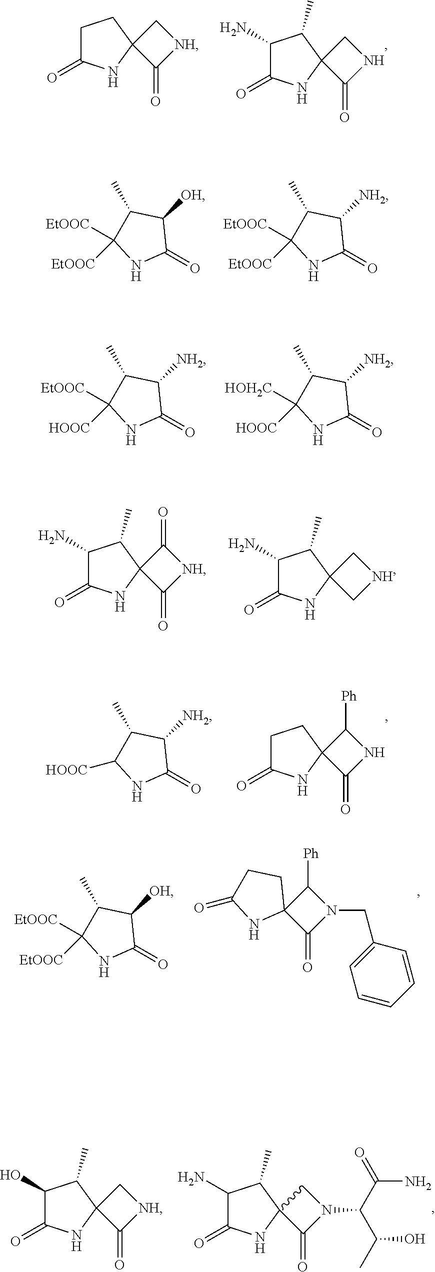

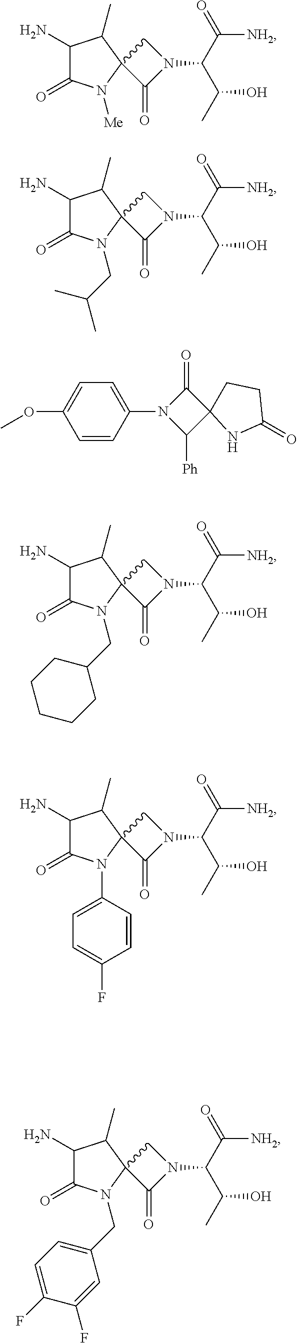

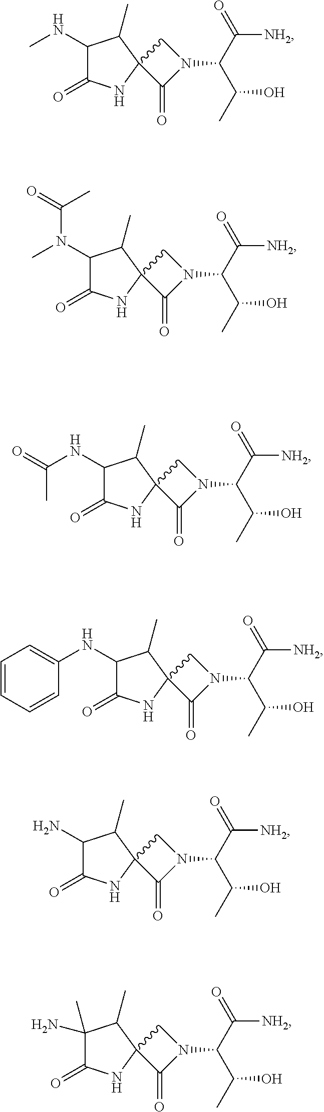 [SMILES: CNC1C(=O)NC2(CN([C@H](C(N)=O)[C@@H](C)O)C2=O)C1C ; CC(=O)N(C)C1C(=O)NC2(CN([C@H](C(N)=O)[C@@H](C)O)C2=O)C1C ; CC(=O)NC1C(=O)NC2(CN([C@H](C(N)=O)[C@@H](C)O)C2=O)C1C ; CC1C(Nc2ccccc2)C(=O)NC12CN([C@H](C(N)=O)[C@@H](C)O)C2=O ; CC1C(N)C(=O)NC12CN([C@H](C(N)=O)[C@@H](C)O)C2=O ; CC1C(C)(N)C(=O)NC12CN([C@H](C(N)=O)[C@@H](C)O)C2=O]

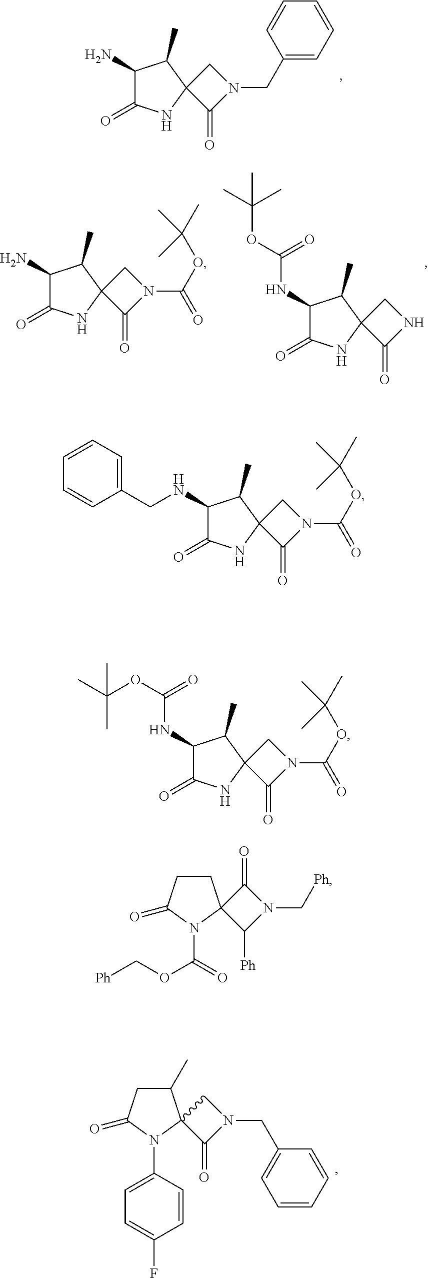

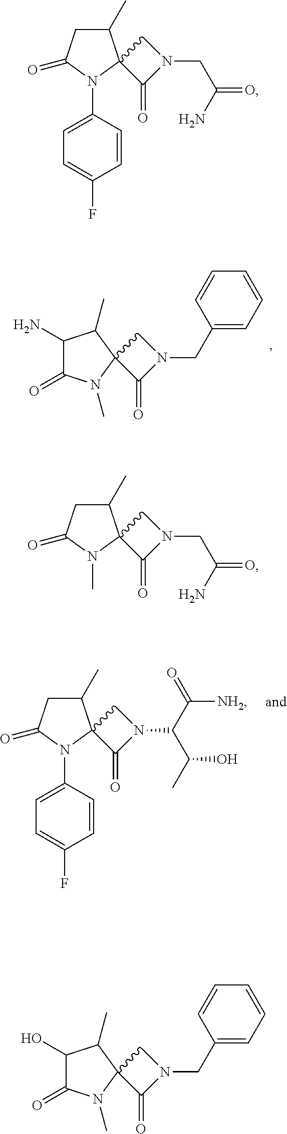

In some embodiments, a contemplated compound for use in a disclosed method is represented by Formula (VII):

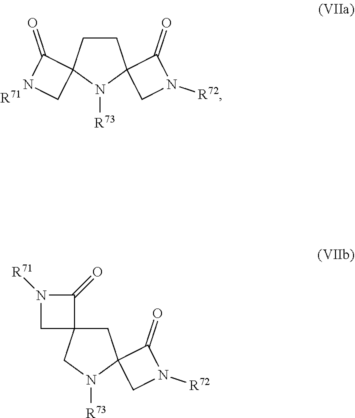

or a pharmaceutically acceptable salt, a stereoisomer, and/or an N-oxide thereof, wherein:
R⁷¹ and R⁷² are independently selected from the group consisting of hydrogen, —C₁-C₆alkyl, —C(O)—C₁-C₆alkyl, —C(O)—O—C₁-C₆alkyl, and —O—CH₂-phenyl;
R⁷³ is selected from the group consisting of hydrogen, —C₁-C₆alkyl, —C(O)—R³¹, and —C(O)—O—R³²; R³¹ is selected from the group consisting of hydrogen, —C₁-C₆alkyl; —C₁-C₆haloalkyl, —C₃-C₆cycloalkyl, and phenyl; R³² is selected from the group consisting of hydrogen, —C₁-C₆alkyl; —C₁-C₆haloalkyl, —C₃-C₆cycloalkyl, and phenyl; wherein any aforementioned C₁-C₆alkyl, independently for each occurrence, is optionally substituted by one, two or three substituents each independently selected from —C(O)NR^{a}R^{b}, —NR^{a}R^{b}, hydroxyl, —SH, phenyl, —O—CH₂-phenyl, and halogen; and any aforementioned phenyl, independently for each occurrence, is optionally substituted by one, two or three substituents each independently selected from —C(O)NR^{a}R^{b}, —NR^{a}R^{b}, —C₁-C₃alkoxy, hydroxyl, and halogen; or R^{a} and R^{b} are each independently for each occurrence selected from the group consisting of hydrogen, —C(O)—O—CH2-phenyl, and —C₁-C₃alkyl; or R^{a} and R^{b} taken together with the nitrogen to which they are attached form a 4-6 membered heterocyclic ring;

In some embodiments, a contemplated compound for use in a disclosed method is:

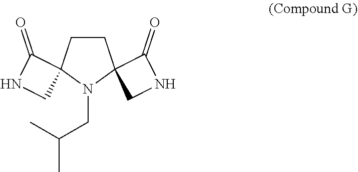 [SMILES: CC(C)CN1[C@@]2(CC[C@@]13CNC3=O)CNC2=O]

In some embodiments, a contemplated compound for use in a disclosed method is:

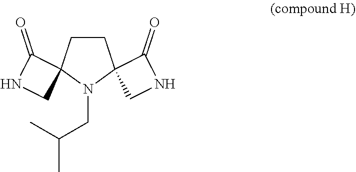 [SMILES: CC(C)CN1[C@]2(CC[C@]13CNC3=O)CNC2=O]

In some embodiments, a contemplated compound for use in a disclosed method is selected from the group consisting of:

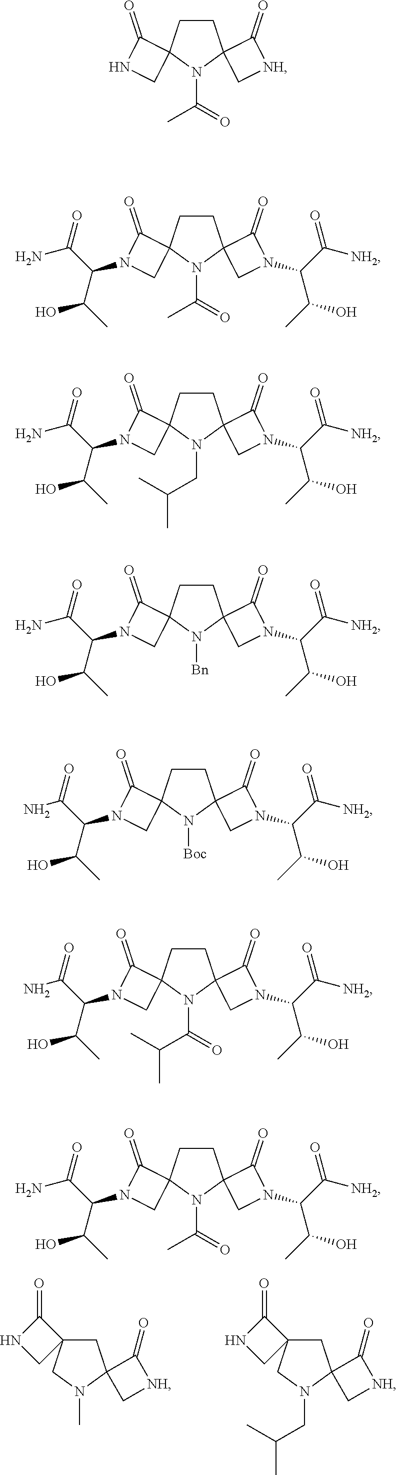

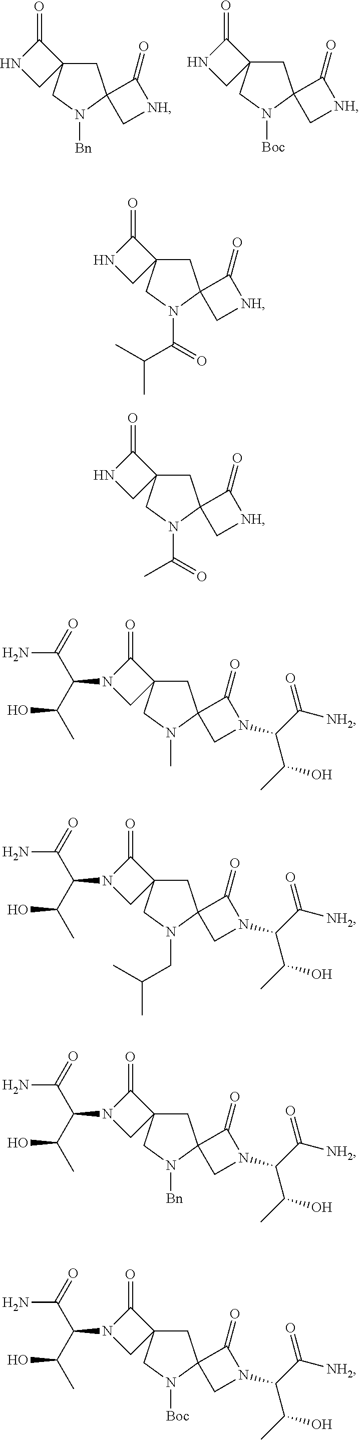

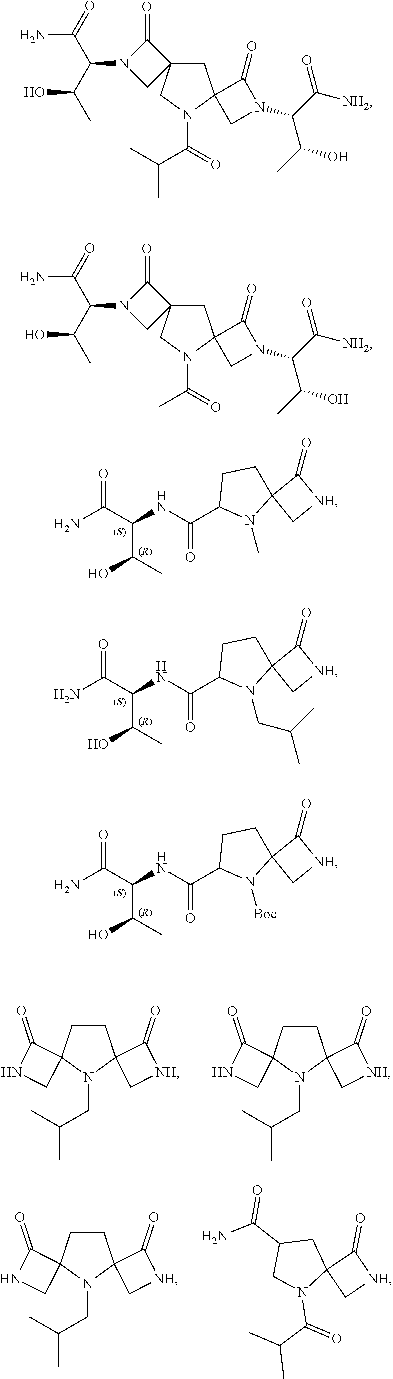

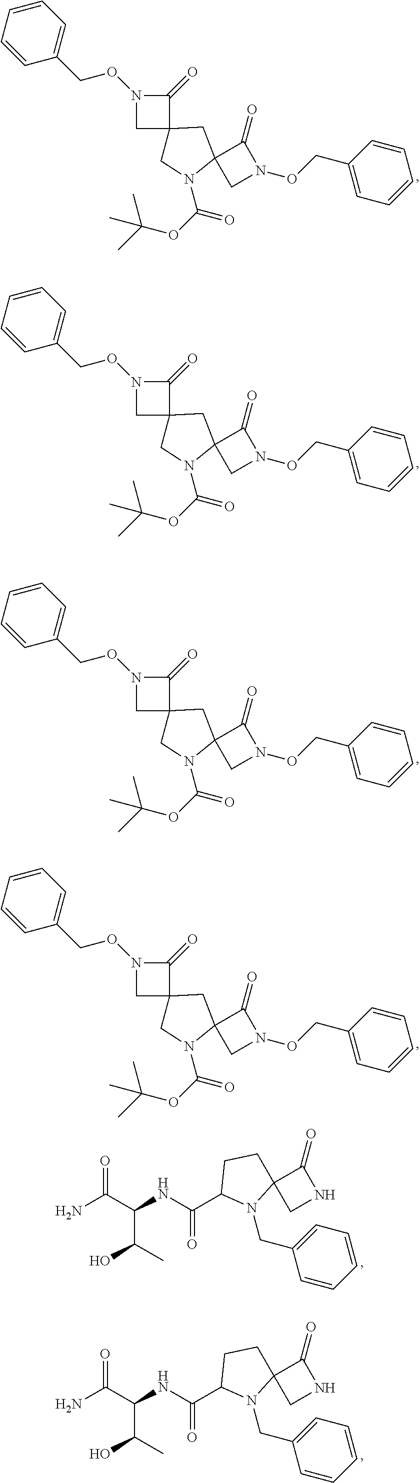

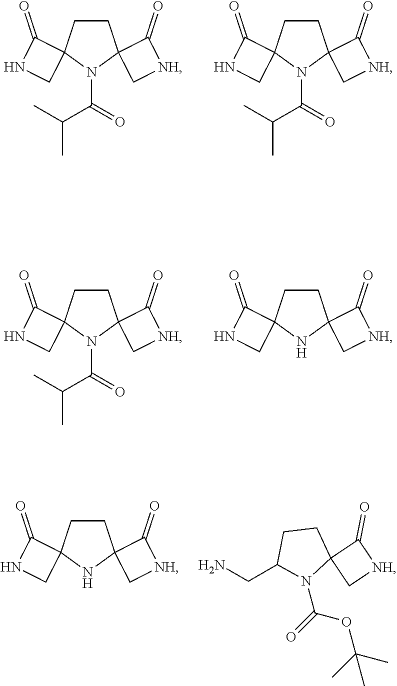 [SMILES: CC(C)C(=O)N1C2(CCC13CNC3=O)CNC2=O ; CC(C)C(=O)N1C2(CCC13CNC3=O)CNC2=O ; CC(C)C(=O)N1C2(CCC13CNC3=O)CNC2=O ; O=C1NCC12CCC1(CNC1=O)N2 ; O=C1NCC12CCC1(CNC1=O)N2 ; CC(C)(C)OC(=O)N1C(CN)CCC12CNC2=O]

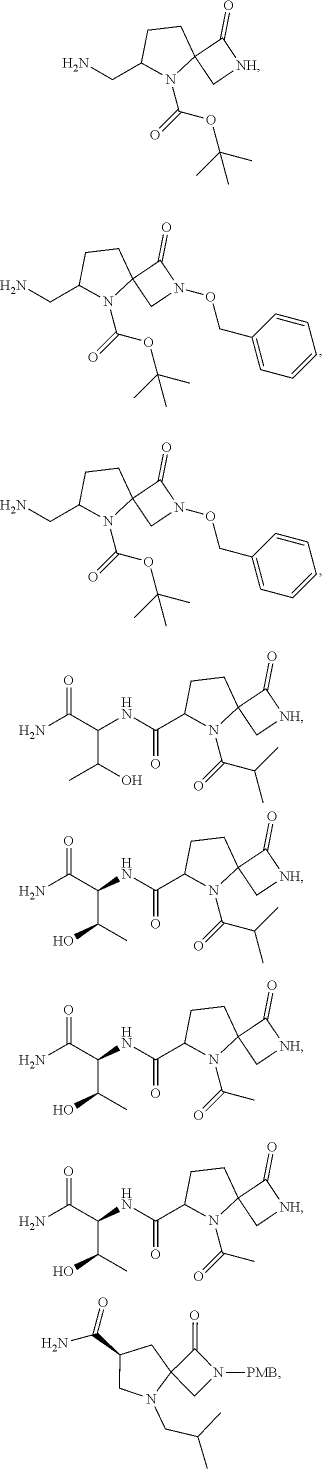

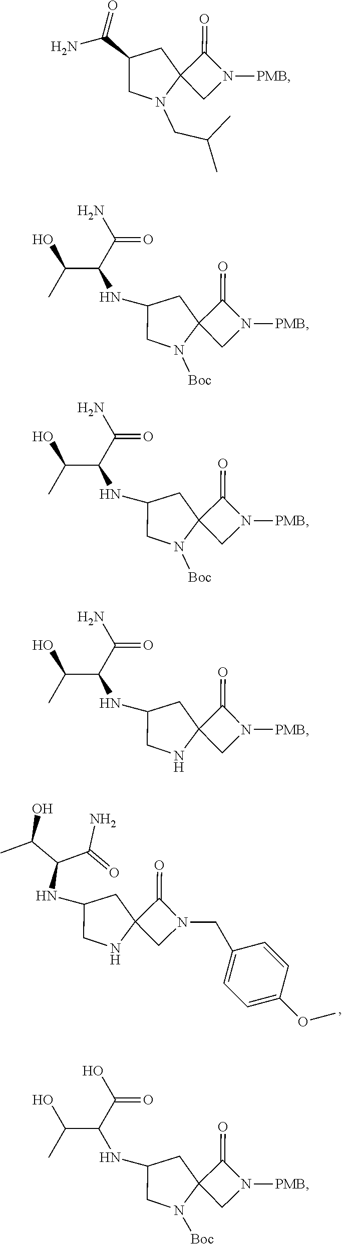

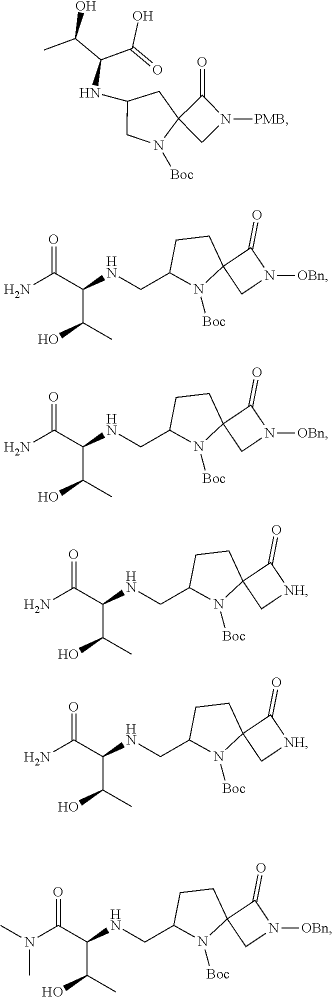

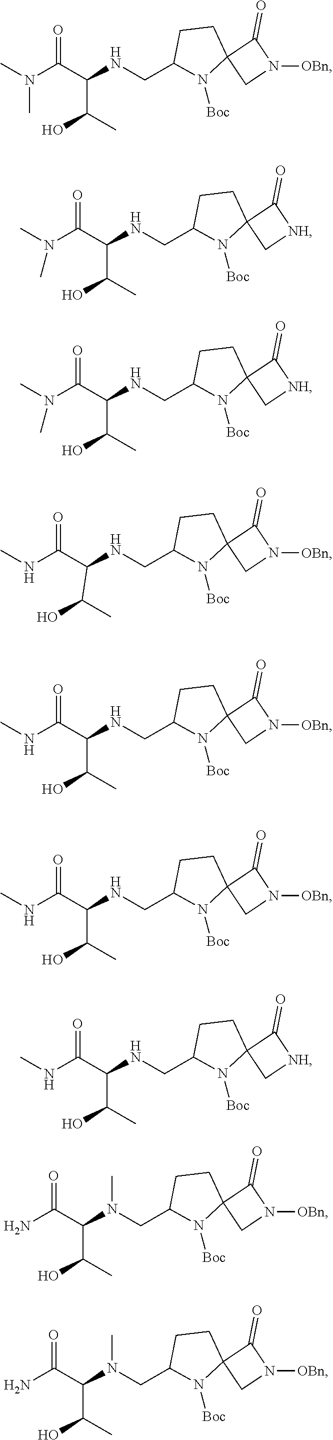

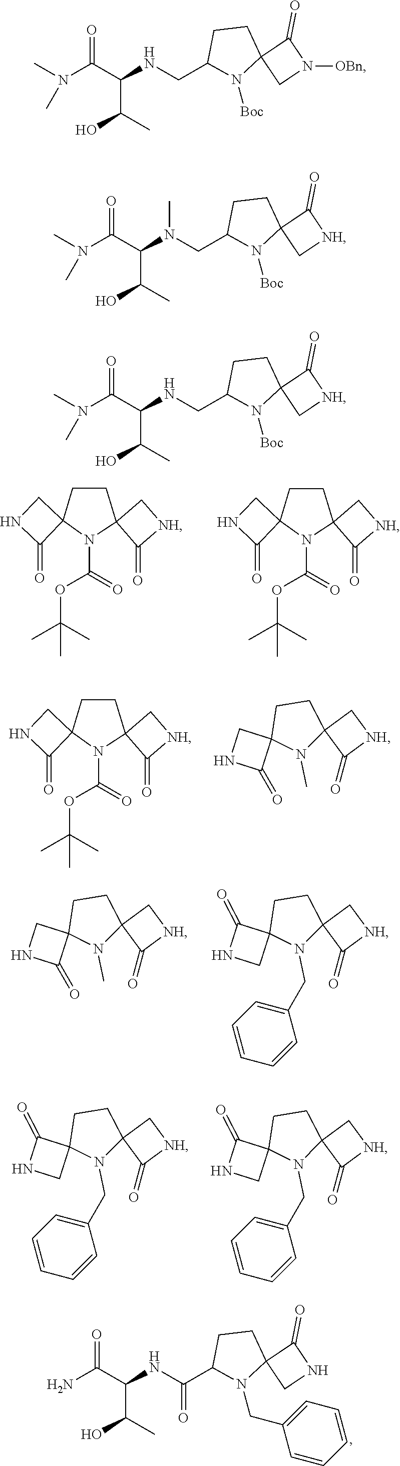

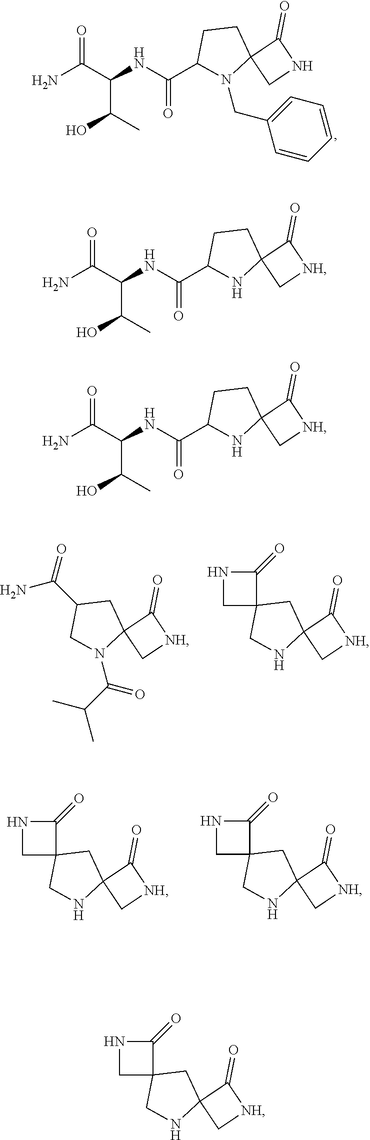

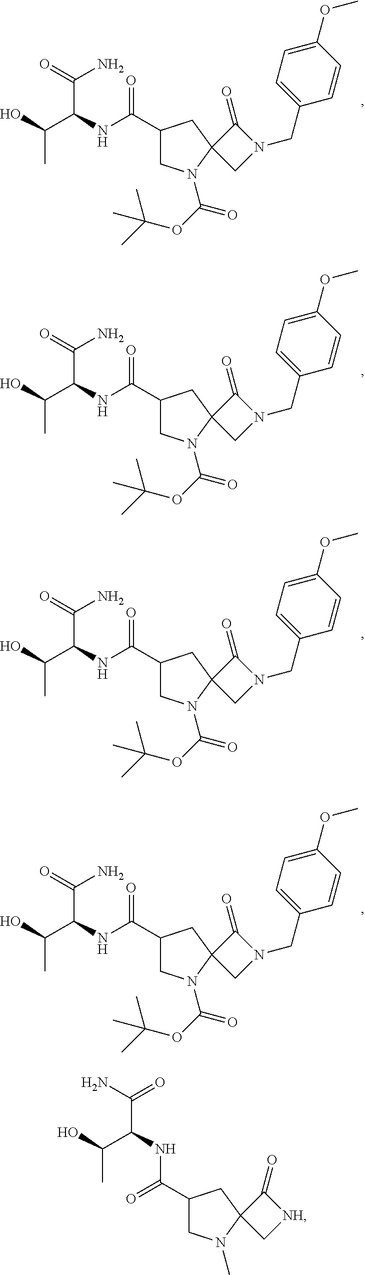

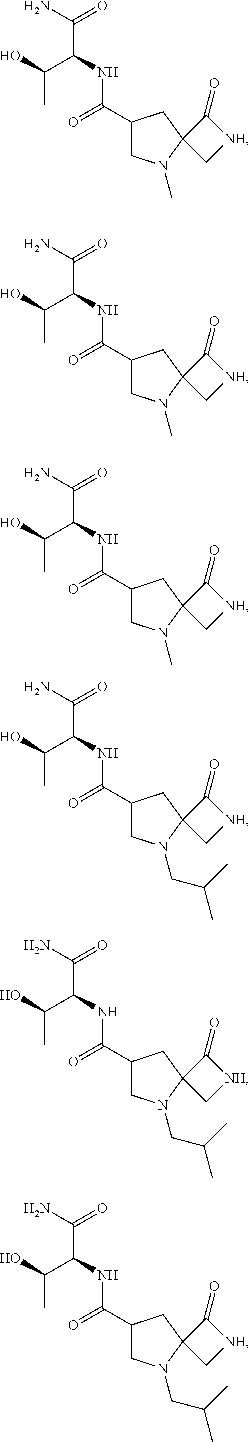 [SMILES: C[C@@H](O)[C@H](NC(=O)C1CN(C)C2(CNC2=O)C1)C(N)=O ; C[C@@H](O)[C@H](NC(=O)C1CN(C)C2(CNC2=O)C1)C(N)=O ; C[C@@H](O)[C@H](NC(=O)C1CN(C)C2(CNC2=O)C1)C(N)=O ; CC(C)CN1CC(C(=O)N[C@H](C(N)=O)[C@@H](C)O)CC12CNC2=O ; CC(C)CN1CC(C(=O)N[C@H](C(N)=O)[C@@H](C)O)CC12CNC2=O ; CC(C)CN1CC(C(=O)N[C@H](C(N)=O)[C@@H](C)O)CC12CNC2=O]

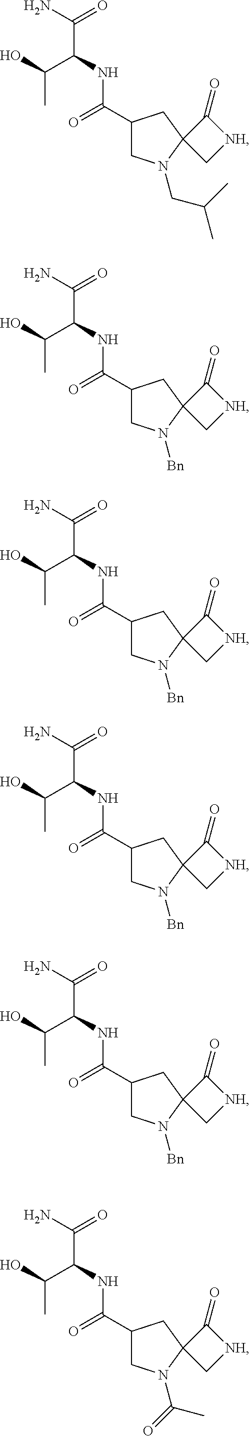 [SMILES: CC(C)CN1CC(C(=O)N[C@H](C(N)=O)[C@@H](C)O)CC12CNC2=O ; C[C@@H](O)[C@H](NC(=O)C1CN(Cc2ccccc2)C2(CNC2=O)C1)C(N)=O ; C[C@@H](O)[C@H](NC(=O)C1CN(Cc2ccccc2)C2(CNC2=O)C1)C(N)=O ; C[C@@H](O)[C@H](NC(=O)C1CN(Cc2ccccc2)C2(CNC2=O)C1)C(N)=O ; C[C@@H](O)[C@H](NC(=O)C1CN(Cc2ccccc2)C2(CNC2=O)C1)C(N)=O ; CC(=O)N1CC(C(=O)N[C@H](C(N)=O)[C@@H](C)O)CC12CNC2=O]

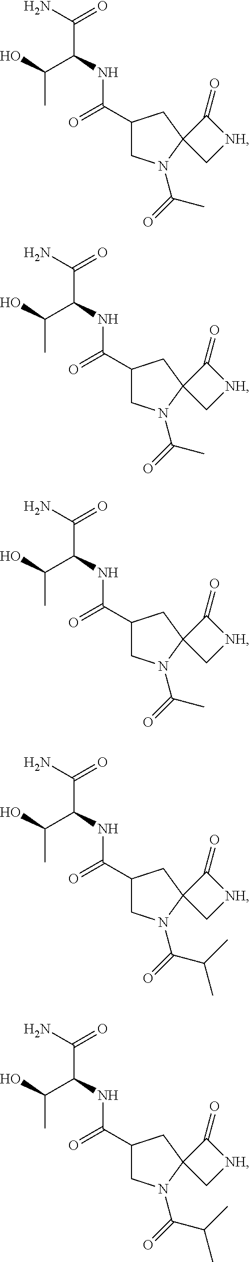 [SMILES: CC(=O)N1CC(C(=O)N[C@H](C(N)=O)[C@@H](C)O)CC12CNC2=O ; CC(=O)N1CC(C(=O)N[C@H](C(N)=O)[C@@H](C)O)CC12CNC2=O ; CC(=O)N1CC(C(=O)N[C@H](C(N)=O)[C@@H](C)O)CC12CNC2=O ; CC(C)C(=O)N1CC(C(=O)N[C@H](C(N)=O)[C@@H](C)O)CC12CNC2=O ; CC(C)C(=O)N1CC(C(=O)N[C@H](C(N)=O)[C@@H](C)O)CC12CNC2=O]

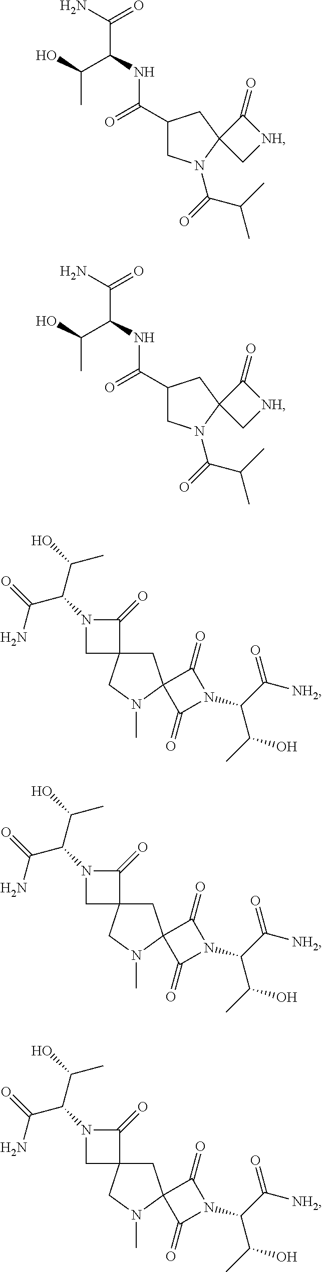 [SMILES: CC(C)C(=O)N1CC(C(=O)N[C@H](C(N)=O)[C@@H](C)O)CC12CNC2=O ; CC(C)C(=O)N1CC(C(=O)N[C@H](C(N)=O)[C@@H](C)O)CC12CNC2=O ; C[C@@H](O)[C@@H](C(N)=O)N1CC2(CN(C)C3(C2)C(=O)N([C@H](C(N)=O)[C@@H](C)O)C3=O)C1=O ; C[C@@H](O)[C@@H](C(N)=O)N1CC2(CN(C)C3(C2)C(=O)N([C@H](C(N)=O)[C@@H](C)O)C3=O)C1=O ; C[C@@H](O)[C@@H](C(N)=O)N1CC2(CN(C)C3(C2)C(=O)N([C@H](C(N)=O)[C@@H](C)O)C3=O)C1=O]

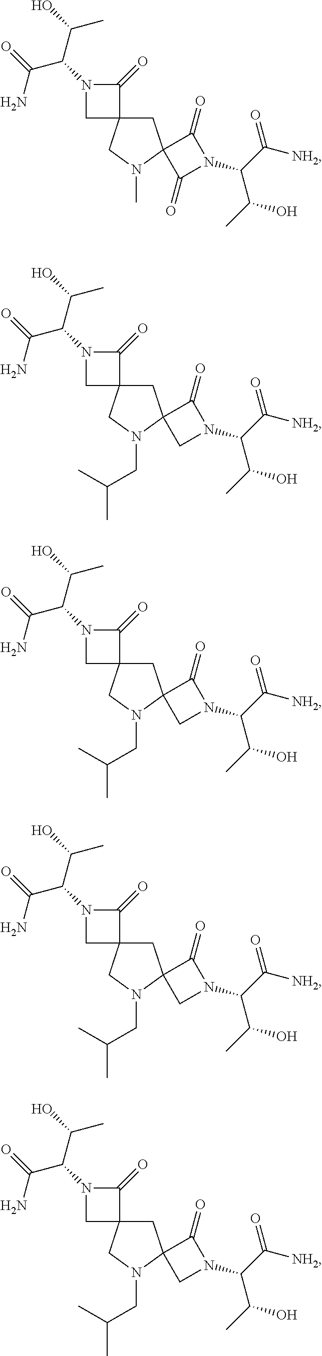

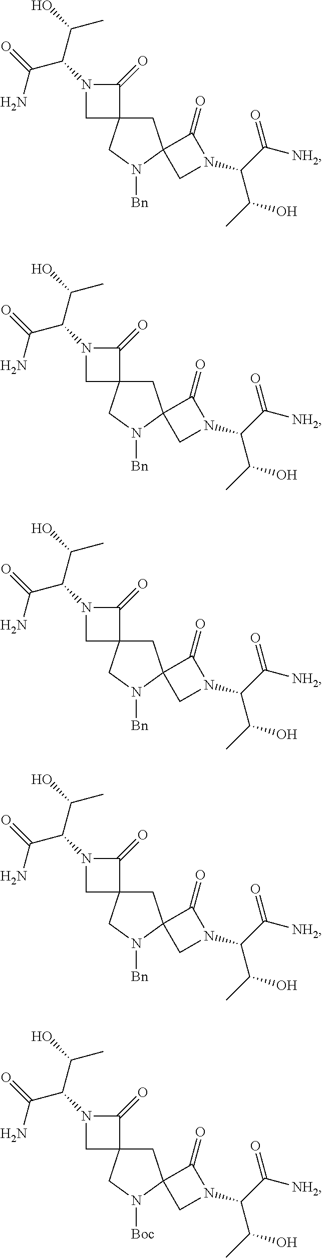 [SMILES: C[C@@H](O)[C@@H](C(N)=O)N1CC2(CN(Cc3ccccc3)C3(CN([C@H](C(N)=O)[C@@H](C)O)C3=O)C2)C1=O ; C[C@@H](O)[C@@H](C(N)=O)N1CC2(CN(Cc3ccccc3)C3(CN([C@H](C(N)=O)[C@@H](C)O)C3=O)C2)C1=O ; C[C@@H](O)[C@@H](C(N)=O)N1CC2(CN(Cc3ccccc3)C3(CN([C@H](C(N)=O)[C@@H](C)O)C3=O)C2)C1=O ; C[C@@H](O)[C@@H](C(N)=O)N1CC2(CN(Cc3ccccc3)C3(CN([C@H](C(N)=O)[C@@H](C)O)C3=O)C2)C1=O ; C[C@@H](O)[C@@H](C(N)=O)N1CC2(CN(C(=O)OC(C)(C)C)C3(CN([C@H](C(N)=O)[C@@H](C)O)C3=O)C2)C1=O]

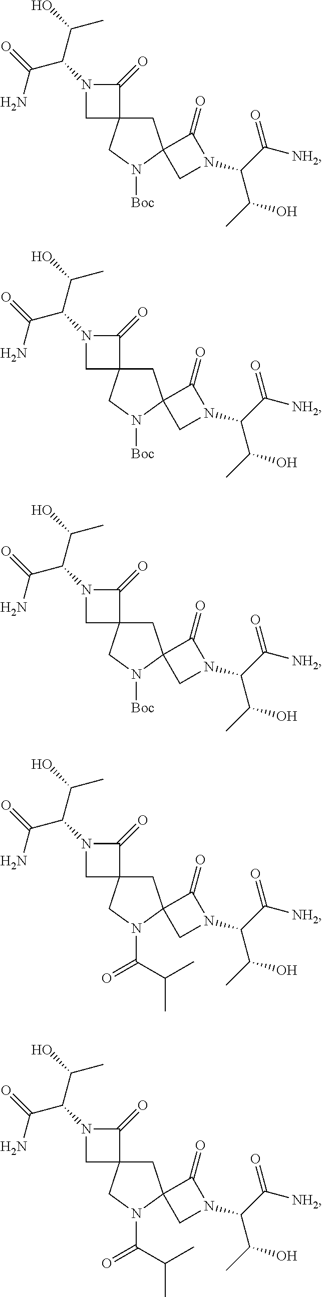 [SMILES: C[C@@H](O)[C@@H](C(N)=O)N1CC2(CN(C(=O)OC(C)(C)C)C3(CN([C@H](C(N)=O)[C@@H](C)O)C3=O)C2)C1=O ; C[C@@H](O)[C@@H](C(N)=O)N1CC2(CN(C(=O)OC(C)(C)C)C3(CN([C@H](C(N)=O)[C@@H](C)O)C3=O)C2)C1=O ; C[C@@H](O)[C@@H](C(N)=O)N1CC2(CN(C(=O)OC(C)(C)C)C3(CN([C@H](C(N)=O)[C@@H](C)O)C3=O)C2)C1=O ; CC(C)C(=O)N1CC2(CN([C@H](C(N)=O)[C@@H](C)O)C2=O)CC12CN([C@H](C(N)=O)[C@@H](C)O)C2=O ; CC(C)C(=O)N1CC2(CN([C@H](C(N)=O)[C@@H](C)O)C2=O)CC12CN([C@H](C(N)=O)[C@@H](C)O)C2=O]

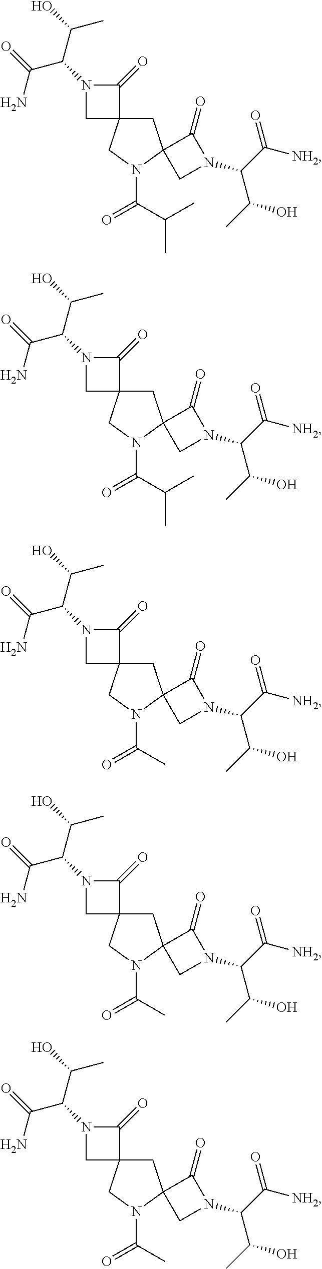 [SMILES: CC(C)C(=O)N1CC2(CN([C@H](C(N)=O)[C@@H](C)O)C2=O)CC12CN([C@H](C(N)=O)[C@@H](C)O)C2=O ; CC(C)C(=O)N1CC2(CN([C@H](C(N)=O)[C@@H](C)O)C2=O)CC12CN([C@H](C(N)=O)[C@@H](C)O)C2=O ; CC(=O)N1CC2(CN([C@H](C(N)=O)[C@@H](C)O)C2=O)CC12CN([C@H](C(N)=O)[C@@H](C)O)C2=O ; CC(=O)N1CC2(CN([C@H](C(N)=O)[C@@H](C)O)C2=O)CC12CN([C@H](C(N)=O)[C@@H](C)O)C2=O ; CC(=O)N1CC2(CN([C@H](C(N)=O)[C@@H](C)O)C2=O)CC12CN([C@H](C(N)=O)[C@@H](C)O)C2=O]

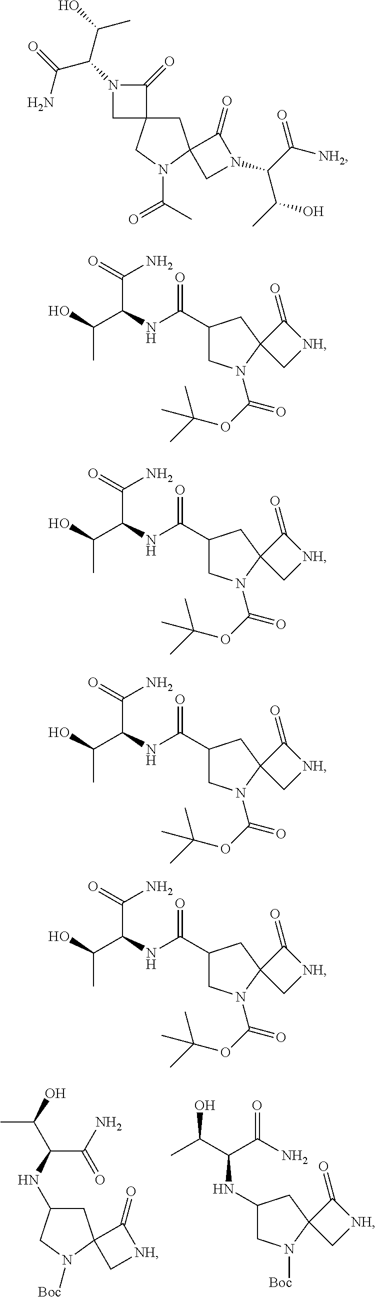

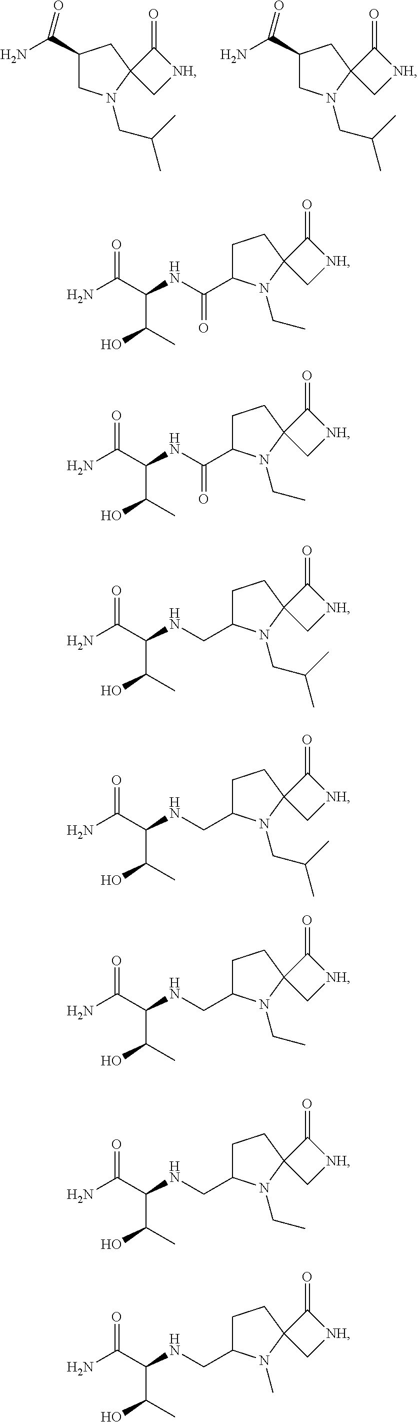

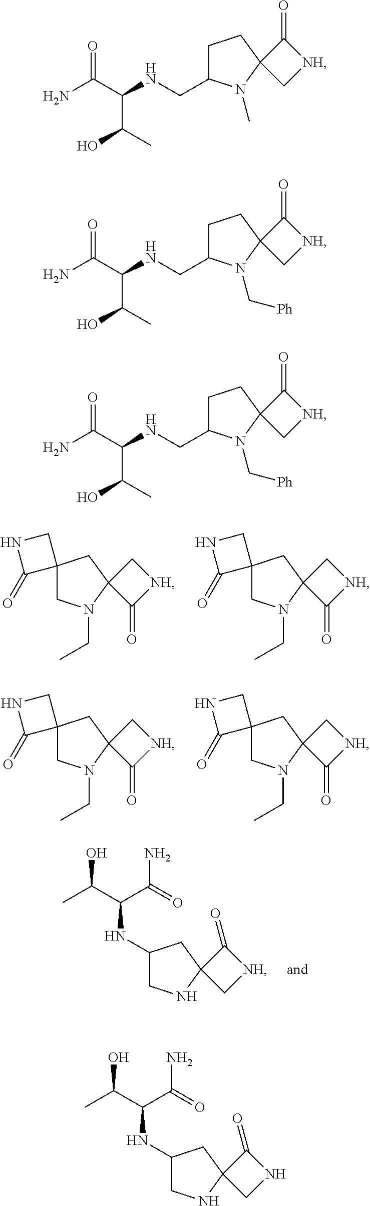

### C. Methods

Disclosed methods for treating a disorder in a patient in need thereof include administering a therapeutically effective amount of a compound described herein or a composition including such a compound. In some embodiments, a disclosed method includes administering a compound to treat patients suffering from a disorder associated with elevated levels of NMDAR antibodies. For example, a contemplated disorder associated with elevated levels of NMDAR antibodies may be paraneoplastic autoimmune encephalitis, non-paraneoplastic autoimmune encephalitis or anti-NMDAR encephalitis.

Anti-NMDAR encephalitis may be characterized by the presence of antibodies against synaptic NMDAR. Patients suffering from anti-NMDAR encephalitis may present varied clinical symptoms. In some embodiments, the anti-NMDAR encephalitis may cause deficits that include, but are not limited to psychiatric and neurological manifestations, autonomic dysregulation, seizures, a decreased level of consciousness, hypoventilation, amnesia, deficits in memory, behavior, and cognition. In some embodiments, the encephalitis is associated with, dysfunction of any part of the brain or spinal cord.

In some embodiments the disorder associated with elevated levels of NMDAR antibodies is immunotherapy-responsive dementia. For example immunotherapy-responsive dementia may include but is not limited to unclassified dementia, progressive supranuclear palsy, corticobasal syndrome, frontotemporal dementia , Lewy body dementia, and primary progressive aphasia.

In some embodiments the disorder associated with elevated levels of NMDAR antibodies is also associated with a tumor (e.g., a benign ovarian or testicular teratoma). In other embodiments the tumor may be cancerous. For example, the tumor may be an ovarian teratoma, a thymic tumor or a testicular tumor. In some embodiments, the cancer associated with the encephalitis is a cervical cancer, head and neck, breast cancer, anogenital, a melanoma, a sarcoma, a carcinoma, lymphoma, leukemia, mesothelioma, glioma, a choriocarcinoma, pancreatic cancer, ovarian cancer or gastric cancer. In some embodiments, the cancer is a carcinomatous lesion of the pancreas. In some embodiments, the cancer is pulmonary adenocarcinoma. In some embodiments, the cancer is colorectal adenocarcinoma. In some embodiments, the cancer is pulmonary squamous adenocarcinoma. In some embodiments, the cancer is gastric adenocarcinoma. In some embodiments, a tumore an ovarian surface epithelial neoplasm (e.g. a benign, proliferative or malignant variety thereof). In some embodiments, the cancer is an oral squamous cell carcinoma. In some embodiments, the cancer is non-small cell lung carcinoma. In some embodiments, the cancer is an endometrial carcinoma. In some embodiments, the cancer is a bladder cancer. In some embodiments, the cancer is a head and neck cancer. In some embodiments, the cancer is a prostate carcinoma. In some embodiments, the cancer is an acute myelogenous leukemia (AML). In some embodiments, the cancer is a myelodysplastic syndrome (MDS). In some embodiments, the cancer is a non-small cell lung cancer (NSCLC). In some embodiments, the cancer is a Wilms' tumor. In some embodiments, the cancer is a leukemia. In some embodiments, the cancer is a lymphoma. In some embodiments, the cancer is a desmoplastic small round cell tumor. In some embodiments, the cancer is a mesothelioma (e.g. malignant mesothelioma). In some embodiments, the cancer is a gastric cancer. In some embodiments, the cancer is a colon cancer. In some embodiments, the cancer is a lung cancer. In some embodiments, the cancer is a breast cancer. In some embodiments, the cancer is a germ cell tumor. In some embodiments, the cancer is an ovarian cancer. In another embodiment, the cancer is a uterine cancer. In another embodiment, the cancer is a thyroid cancer. In some embodiments, the cancer is a hepatocellular carcinoma. In some embodiments, the cancer is a thyroid cancer. In some embodiments, the cancer is a liver cancer. In some embodiments, the cancer is a renal cancer. In some embodiments, the cancer is a Kaposi's sarcoma. In some embodiments, the cancer is a sarcoma. In some embodiments, the cancer is another carcinoma or sarcoma.

Methods disclosed herein, e.g., of treating a disorder associated with elevated NMDAR antibodies may further comprise identifying the patient as having NMDAR IgA, IgD, IgE, IgG, and IgM isotype antibodies, e.g., NMDAR IgG type antibodies.

Anti-NMDAR encephalitis may be diagnosed by tests that include but are not limited to the detection of anti-NMDAR antibodies in the serum or CSF of patients. Patient serum and CSF may be tested for reactivity with the hippocampal tissue on rat brain sections, cell-surface labeling of cultured hippocampal neurons, or tested for reactivity with NR1/NR2 transfected human embryonic kidney (HEK) cells. The CSF may also present pleocytosis, increased protein concentration, oligoclonal bands and high IgG index. Other diagnostic tests may include electroencephalogram and MRI tests. Abnormal MRIs commonly show T2 or FLAIR hyperintensities in cortical or subcortical brain regions, sometimes with mild or transient contrast enhancement. Abnormal EEGs show slow and disorganized activity in the delta/theta range, sometimes with superimposed electrographic seizures.

Pharmaceutical compounds and/or compositions may be assayed for effectiveness in restoring NMDA receptor and/or NMDA receptor subtype surface expression.

For example, compounds may be evaluated in assays, for their ability to restore cell surface expression of the NR2B subunit of the NMDAR receptor following exposure to antibodies from ANRE patient serum or CSF. For example, assays may also measure a compounds modulation of NMDAR1 antibody mediated activity in hippocampal slices. Contemplated methods may include, but are not limited administration of compounds A, B, C,D or E.

### EXAMPLES

### Example 1. Assaying Compound A, B, and C for Restoration of Surface NR2B Expression in Cells Treated With Purified ANRE Patient Serum IgG

This example demonstrates a β-lactamase assay that may be used to determine the effect of each of compounds A, B, and C on NR2B surface expression following incubation with purified ANRE patient serum IgG antibodies. 1 pM of compound A (in the absence of Ab) served as a positive control to demonstrate ˜20% increase in cell surface NR2B expression. Un-transfected (Untfx) cells were used as a negative control for surface β-lactamase activity.

hNR1/PSD95/NR2B-expressing HEK cells tagged with a β-lactamase enzyme at the N-terminus of NR2B are incubated with a 1:50 dilution of purified patient serum IgG for 45 min. The cells are washed with buffer to remove antibodies and then incubated for 15 min in the presence or absence of each of compounds A, B, or C at concentrations of 10 fM, 1 pM, 100 pM and 10 nM respectively. After said time, the membrane impermeable substrate nitrocefin and ±each of compounds A, B, and C are added. The NR2B N-terminal β-lactamase cleaves nitrocefin, resulting in a change of absorbance to 486 nm. The absorbance at 486 nm is measured every minute for 30 min.

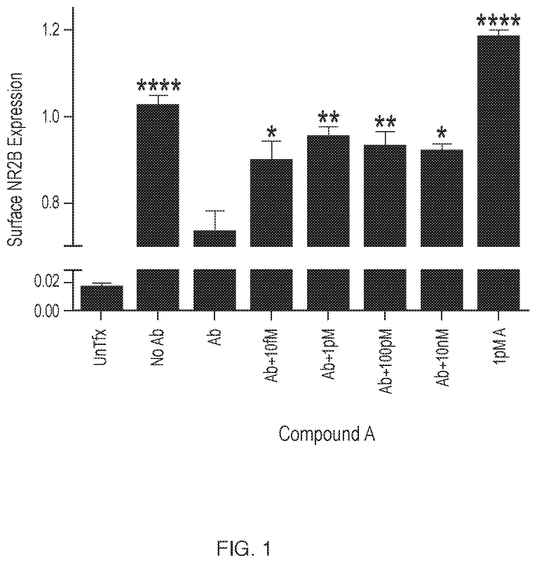

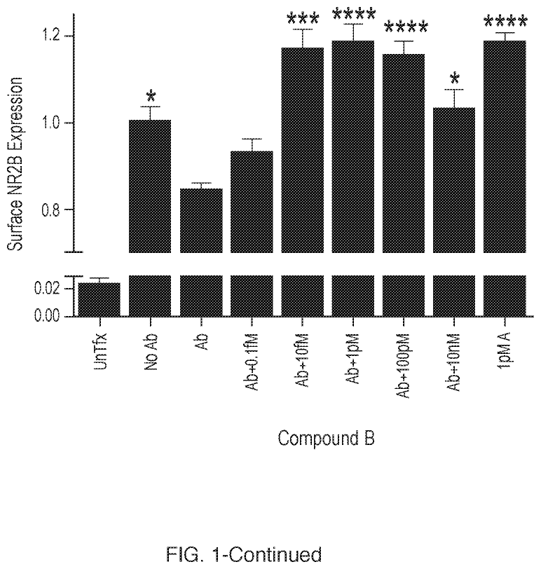

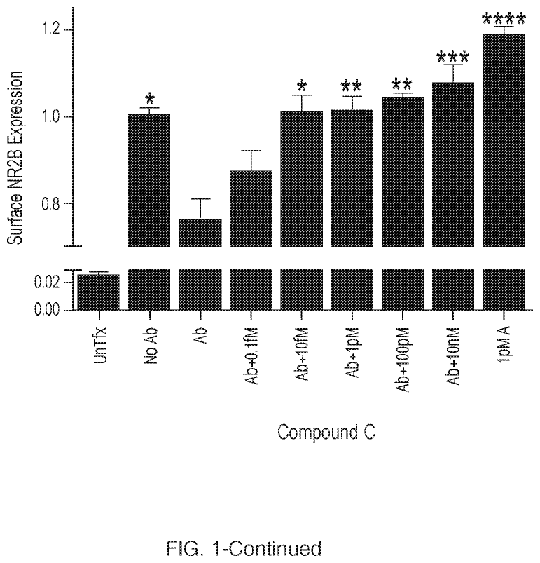

Slopes of optical density are graphed as fold change over the vehicle. N=8; Statistics were determined by one-way ANOVA compared to antibody only (Ab). * p≤0.05, ** p≤0.01, *** p≤0.001, **** p≤0.0001 (FIG. 1).

Incubation of NR2B expressing cells with purified ANRE patient serum IgG decreases cell surface NR2B expression compared to “No Ab” controls (FIG. 1).

Following incubation with patient-derived antibodies, each of Compound A, B, and C restore NR2B surface expression to levels observed in the “No Ab” control (FIG. 1).

### Example 2. Assaying Compound A, B, and C for Restoration of Surface NR2B Expression in Cells Treated With ANRE Patient CSF

This example demonstrates a β-lactamase assay used to determine the effect of each of compounds A, B, and C on NR2B surface expression following incubation with ANRE patient CSF. 1 pM of compound A (in the absence of Ab) served as a positive control.

In this example hNR1/PSD95/NR2B expressing HEK cells tagged with a β-lactamase enzyme at the N-terminus of NR2B are incubated with CSF (1:10 dilution) from each of three individual ANRE patients (N1, N2, and N3).

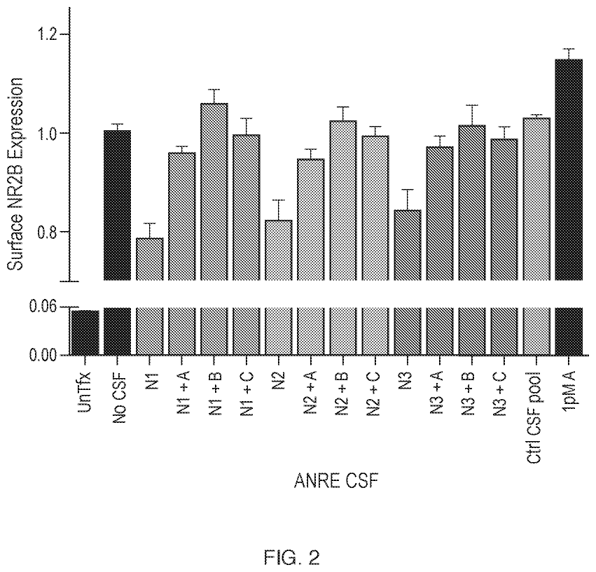

The surface NR2B expression of cells exposed to CSF from each individual ANRE patient (N1, N2, and N3) decreased compared to ‘no CSF’ controls (N=12) or to a pool of 7 individual CSF samples from non-ANRE patients (FIG. 2).

Following incubation with CSF from each individual ANRE patient (N1, N2, and N3) the addition of each of compounds A. B, and C each restored cell surface NR2B expression to vehicle levels (FIG. 2).

### Example 3. Assaying Compounds A-H for Restoration of Surface NR2B Expression in Cells Treated With Purified ANRE Patient Serum IgG

This example demonstrates a β-lactamase assay that may be used to determine the effect of each of compounds A, B, C, D, E, F, and H on NR2B surface expression following incubation with purified ANRE patient serum IgG. 1 pM of compound A served as a positive control to demonstrate ˜20% increase in cell surface NR2B expression. Un-transfected (Untfx) cells were used as a negative control for surface β-lactamase activity. Human IgG (Sigma-Aldrich) at a concentration of 0.5 mg/ml was used as a negative control for non-specific receptor internalization.

hNR1/PSD95/NR2B expressing HEK cells tagged with a β-lactamase enzyme at the N-terminus of NR2B are incubated with purified patient serum IgG (1:50 dilution) for 45 min. The cells are washed to remove antibodies and then incubated for 15 min in the presence or absence of each of compounds A, B, C, D, E, F, G, and H at concentrations of 10 fM, 1 pM, 100 pM and 10 nM respectively. After said time, nitrocefin in buffer and ±each of compounds A-H are added and the absorbance at 486 nm is measured every minute for 30 min.

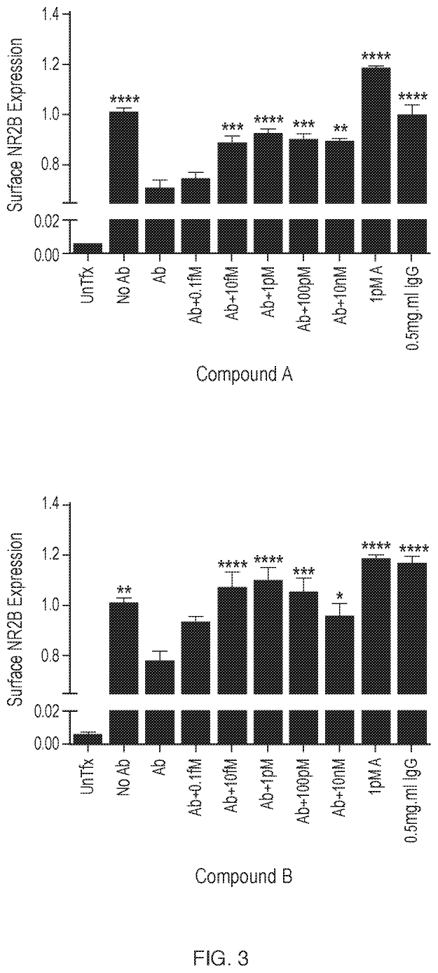

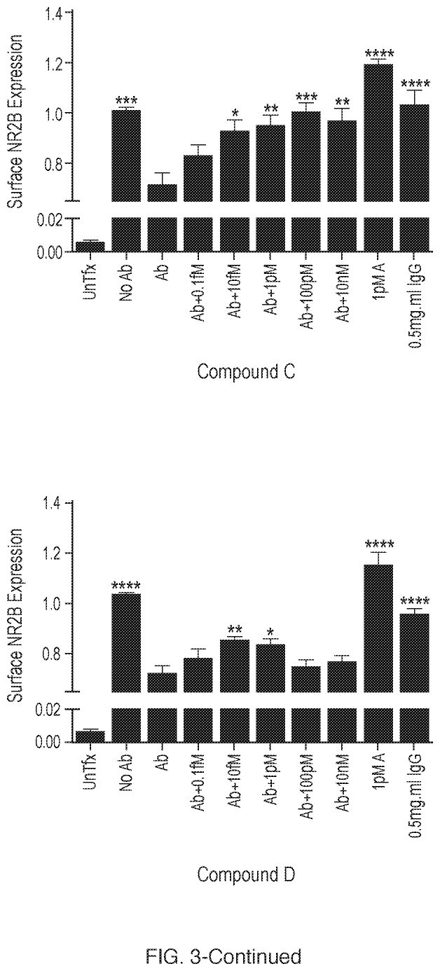

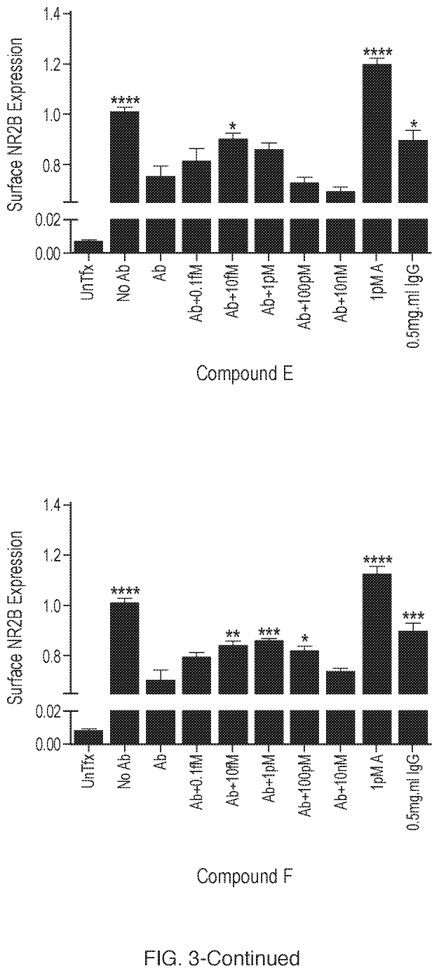

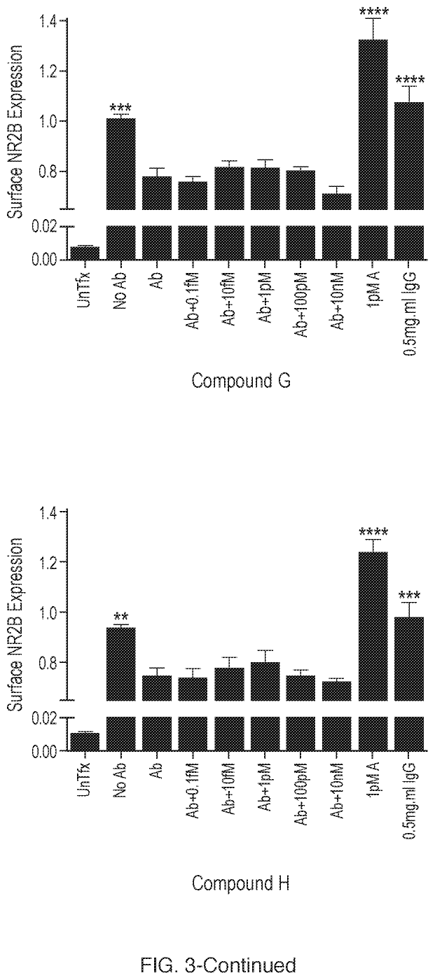

Slopes of optical density are graphed as fold change over the vehicle. N=8-12; Statistics were determined by one-way ANOVA compared to antibody only (Ab). * p≤0.05, ** p≤0.01, *** p≤0.001, **** p≤0.0001 (FIG. 3).

Incubation of NR2B expressing cells with purified ANRE patient serum IgG (Ab) decreases cell surface NR2B expression compared to “No Ab” control (FIG. 3).

Following incubation with patient-derived antibodies, Compound A, B, and C restore NR2B surface expression to levels observed in the “No Ab” control (FIG. 3).

Following incubation with patient-derived antibodies, Compound D, E, F, G, and H failed to restore NR2B surface expression to levels observed in the “No Ab” control (FIG. 3).

### Example 4. Recovery of NR2B Surface Expression Following Antibody Incubation

This example shows a β-lactamase assay that may be used to determine NR2B surface expression in hNR1/PSD95/NR2B expressing HEK cells following antibody incubation.

hNR1/PSD95/NR2B expressing HEK cells tagged with a β-lactamase enzyme at the N-terminus of NR2B are incubated with purified patient serum IgG (1:50 dilution) for 45 min. After said time, nitrocefin is added and the absorbance at 486 nm is measured at 0 min, 15 min, 30 min, 45 min, 60 min, 2 h, 4 h, 6 h, and 24 h time points following substrate administration.

Slopes of optical density are graphed as fold change over the vehicle. Statistics are calculated by ANOVA compared to antibody only (Ab). ** p≤0.01, **** p≤0.0001.

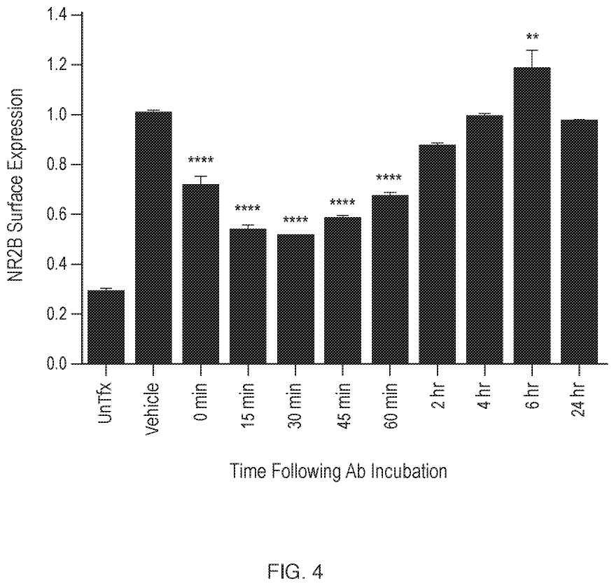

In the absence of drug, NR2B cell surface expression returns to basal levels by 2-4 hours post antibody incubation (FIG. 4).

### Example 5. Compound A Induced Rescue of Schaffer Collateral-CA1 LTP From Acute NR1 Antibody-Induced Effects

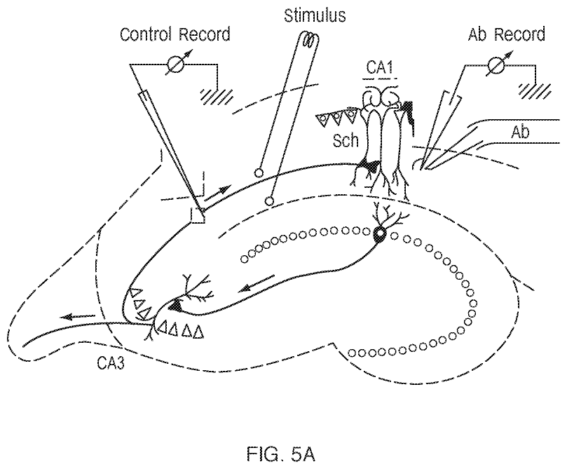

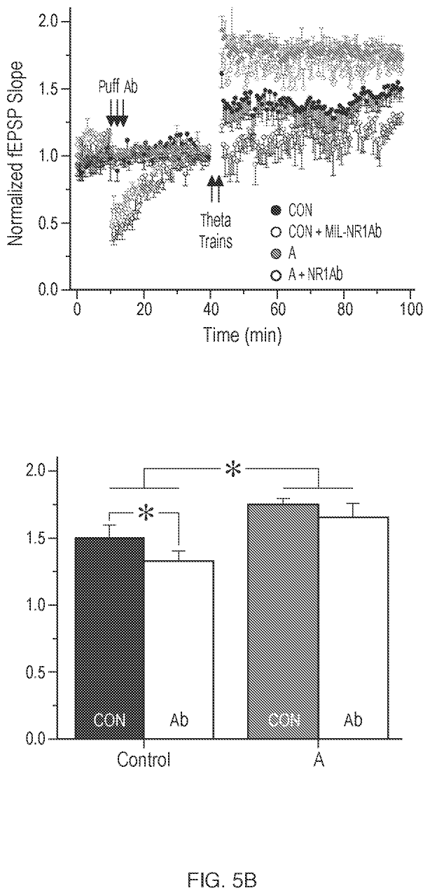

This example shows a normalized field excitatory postsynaptic potential (fEPSP) Slope as a function of time (min) and a summary bar graph from experiments assessing the effects of anti-NR1 antibodies and Compound A on hippocampal slice long-term potentiation (LTP) using the configuration in FIG. 5A. Two extracellular recording patch pipettes were placed at equal distances (˜500 μm) on either side of a single bipolar stainless steel stimulating electrode (Frederick Haer Inc.). A single test stimulus was applied each 30 seconds, to evoke field excitatory postsynaptic potentials (fEPSPs) recorded simultaneously at the Control and Antibody (Ab) recording sites. An additional patch pipette filled with artificial cerebrospinal fluid containing a 1:20 dilution of commercial NR1 antibody (Millipore) was placed within 50 μm of the Ab recording site at the same depth as the recording electrode (50-100 μm), and multiple brief (50-100 ms; Picospritzer, General Dynamics) pressure puffs were used to apply Ab focally to the synapses only at the Ab recording site. Compound A5 (500 nM) was bath-applied 10 minutes prior to the application of three puffs of Ab, and remained in the bath throughout the experiment.

LTP was induced by the stimulation of Schaffer collateral axons with high frequency theta burst stimulus trains. The fEPSP slope was measured before and after the induction of LTP in hippocampal slices treated with control, control +NR1Ab, Compound A, or Compound A +NR1Ab.

As shown in FIG. 5B, NR1 antibodies reduced the magnitude of LTP compared to the control population after high frequency stimulation of rat Schaffer collateral-evoked NMDA fEPSPs recorded in CA1 pyramidal neurons.

As shown in FIG. 5B, Compound A increased the magnitude of LTP relative to a control population after high frequency stimulation of rat Schaffer collateral-evoked NMDA fEPSPs recorded in CA1 pyramidal neurons.

As shown in FIG. 5B, Compound A rescues Schaffer collateral-CA1 LTP from acute NMDAR1 antibody (1:20 dilution) effects.

### Example 6. NMDAR 2B Subunit Trafficking in Wild-Type and Mutant R393A Receptors

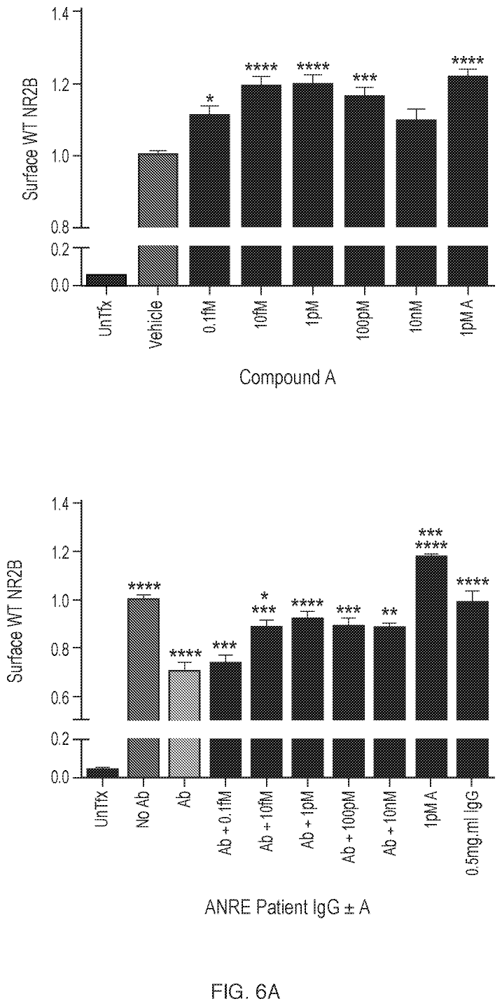

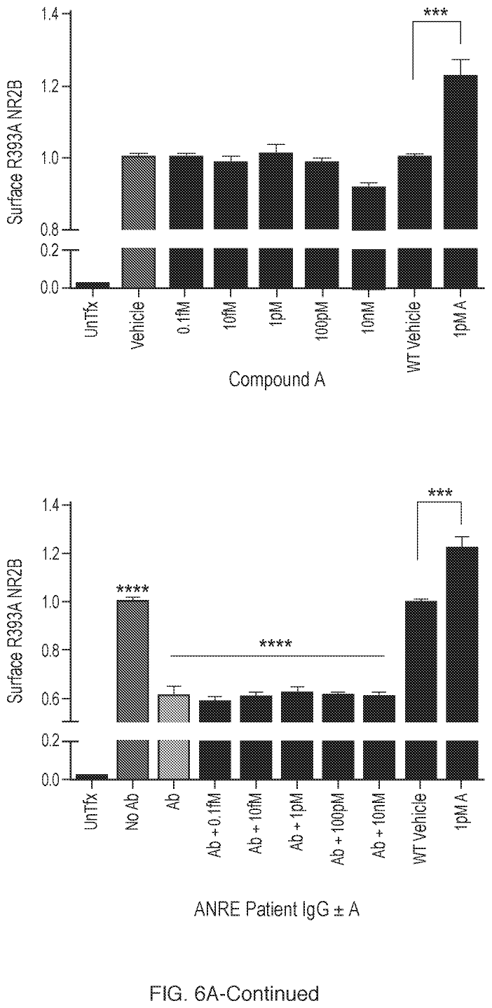

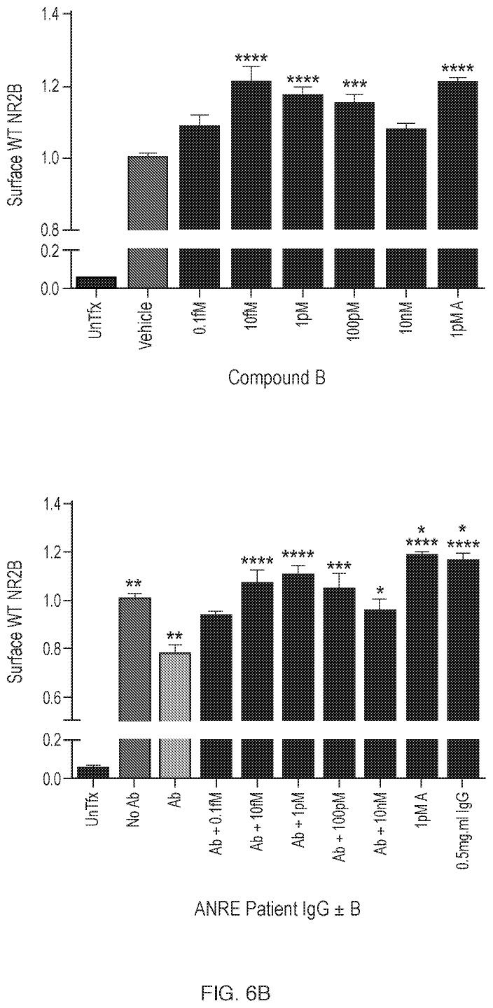

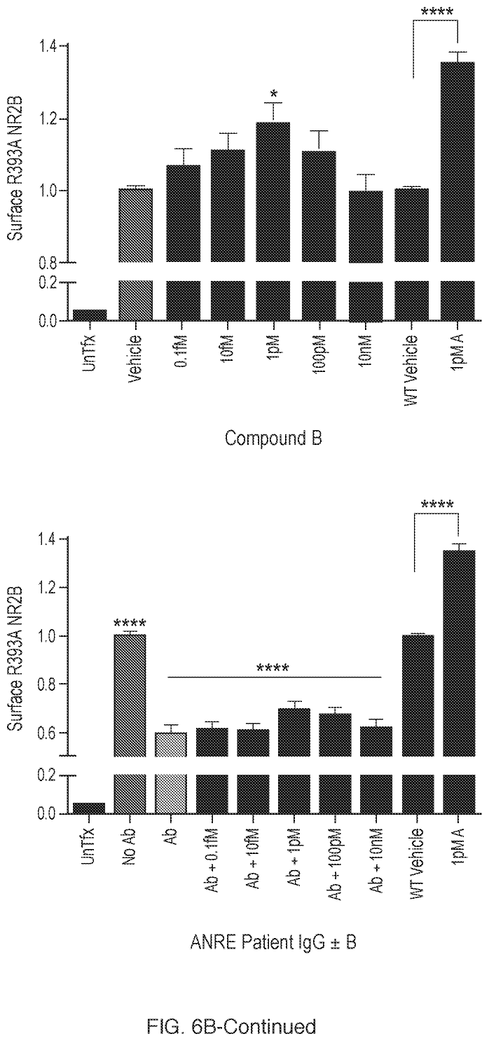

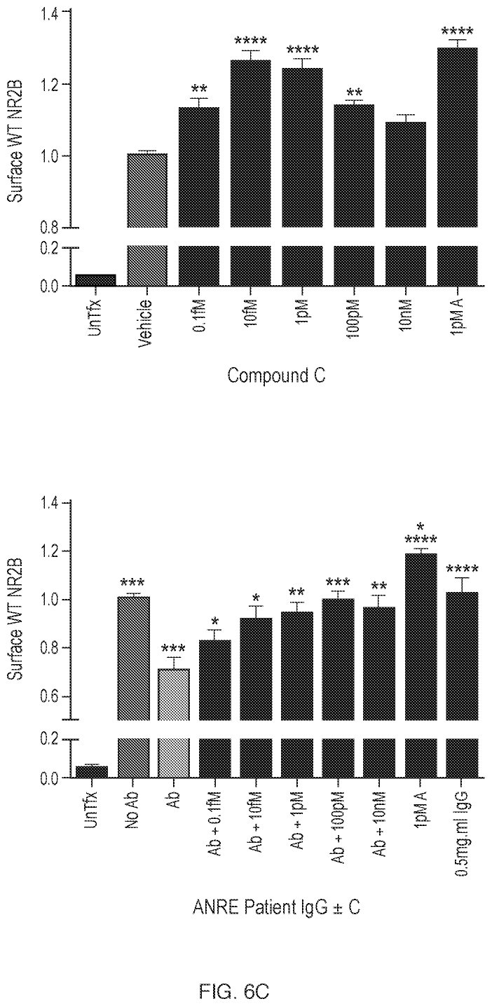

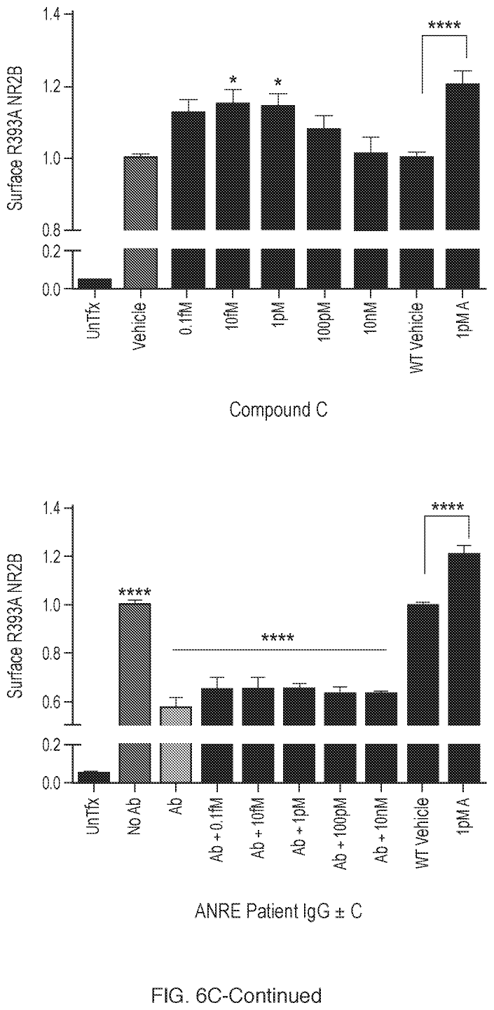

This example demonstrates a β-lactamase assay that may be used to determine the effect of varying concentrations of each of compounds A (FIG. 6A), B (FIG. 6B), and C (FIG. 6C) on NR2B surface expression in wild-type and NR2B:R393A mutant receptors following incubation with or without ANRE patient serum IgG antibodies. 1 pM of compound A (in the absence of Ab) served as a positive control to demonstrate ˜20% increase in wild-type cell surface NR2B expression. Un-transfected (Untfx) cells were used as a negative control for surface β-lactamase activity. Human IgG (Sigma-Aldrich) at a concentration of 0.5 mg/ml was used as a negative control for non-specific receptor internalization.

Each of compounds A (FIG. 6A), B (FIG. 6B), and C (FIG. 6C) increase surface NR2B expression in wild-type NR2B receptors relative to untreated vehicle controls.

Incubation of wild-type NR2B expressing HEK cells with purified ANRE patient serum IgG decreases surface NR2B expression compared to “No Ab” controls. Following incubation with patient-derived antibodies, each of compounds A (FIG. 6A), B (FIG. 6B), and C (FIG. 6C) restore NR2B surface expression to levels observed in the “No Ab” controls.

Mutation at NR2B:R393A abolishes the ability of each of compounds A (FIG. 6A), B (FIG. 6B), and C (FIG. 6C), to restore cell surface NR2B expression showing that this amino acid is a critical determinant within the binding site for each of compounds A, B, and C.

Mutation at NR2B:R393A abolishes the ability of each of compounds A (FIG. 6A), B (FIG. 6B), and C (FIG. 6C), to restore cell surface NR2B expression following exposure to ANRE patient serum IgG.

### Methods

### Creation of the Stable hGluN1/PSD-95 Expressing HEK Cell Line

A HEK293 cell line stably expressing hGluN1 was created as previously described (Khan et al, 2018). The cDNA encoding human PSD-95 (GenBank NM_001365) was PCR amplified from OriGene clone #SC303004 and sub-cloned into the pTRE2pur vector. The hGluN1 expressing HEK cells were then transfected with human PSD-95/pTRE2pur vector using X-tremeGENE9 transfection reagent. Stable PSD-95 clones were selected in media containing puromycin.

### Creation of a hGluN2B Vector and N-Terminal β-lactamase Construct

The hGluN2B vector was constructed as previously described (Khan et al, 2018). The β-lactamase (GenBank NC_0051248) construct was synthesized by Integrated DNA Technologies, Inc. and was subcloned immediately following the signaling peptide at the N-terminus of the hGluN2B sequence using standard molecular techniques.

### Purification of Human Anti-NMDA Receptor Encephalitis IgG Antibodies

Patient plasma was rapidly thawed at 37° C. and mixed with antibody binding buffer (Pierce, 54200) at a ratio of 2:1. IgG was purified by addition of protein A/G plus agarose (Pierce, 20423) at a ratio of 25 μl/ml serum and incubated at 4° C. overnight with agitation. Agarose beads were collected by centrifugation at 1,000×g for 1 min and washed extensively with PBS at 4° C. Antibody-bound beads were then transferred to a spin cup (Pierce, 69702) and excess PBS removed by centrifugation at 10,000×g for ˜1 min. IgG was eluted with 200 mM glycine (pH 2.5) into 1M Tris (pH 8.5) for neutralization at a ratio of 10:1 by centrifugation at 10,000×g. Eluted IgG fraction was then transferred to a dialysis cassette (Pierce, 66380) and dialyzed against PBS at 4° C. for at least three rounds with 1000× dilution factor/round to remove glycine. Purified antibody was concentrated via 100,000 NMWL protein concentrator (Amicon, UFC510096) to a volume equal to that of the wet agarose beads. IgG fraction purity was assessed via Coomassie stain and compared to normal human IgG (Sigma, 14506) for quality control prior to titering and use in the β-lactamase assay.

### β-lactamase Assay

HEK293 cells stably expressing human GluN1 and PSD-95 were transiently transfected with hGluN2B tagged at the N-terminus with the β-lactamase enzyme using X-tremeGENE9 transfection reagent (Sigma-Aldrich). Transfected cultures were maintained in media containing ketamine (0.18 mg/ml) to minimize excitotoxicity. Twenty-four hours after transfection, cells were re-plated into poly-d-lysine coated 96-well plates at a concentration of 30,000 cells/well. The following day, cells were washed with buffer (HBSS, 10 mM HEPES, 50 mM glutamate) and then incubated with a 1:50 dilution of purified patient IgG for 45 min at 37° C. The cells were washed once with buffer and then incubated for 15 min at 37° C. with buffer ±each of compound A, B and C. Nitrocefin in buffer (100 mM final; Cayman Chemical) ±each of compound A, B and C was added to each well and absorbance at 486 nm was measured every minute for 30 min at 37° C. in a plate reader. Un-transfected (Untfx) cells were used as a negative control for surface β-lactamase activity. Human IgG (Sigma-Aldrich) at a concentration of 0.5 mg/ml was used as a negative control for non-specific receptor internalization. Slopes of optical density are graphed as fold change over vehicle, mean ±SEM (n=8-12). Statistics were determined by one-way ANOVA compared to antibody only (Ab).

### Hippocampal Slice Protocols

The experimental protocol to test the effects of focal application of NR1 antibodies on synaptic transmission and long-term potentiation (LTP) of synaptic strength at Schaffer collateral-CA1 synapses in hippocampal slices in vitro is illustrated in FIG. 5A below. Two extracellular recording patch pipettes were placed at equal distances (˜500 μm) on either side of a single bipolar stainless steel stimulating electrode (Frederick Haer Inc.). A single test stimulus was applied each 30 seconds, to evoke field excitatory postsynaptic potentials (fEPSPs) recorded simultaneously at the Control and Antibody (Ab) recording sites. An additional patch pipette filled with artificial cerebrospinal fluid containing a 1:20 dilution of commercial NR1 antibody (Millipore) was placed within 50 μm of the Ab recording site at the same depth as the recording electrode (50-100 μm), and multiple brief (50-100 ms; Picospritzer, General Dynamics) pressure puffs were used to apply Ab focally to the synapses only at the Ab recording site. Compound A (500 nM) was bath-applied 10 minutes prior to the application of three puffs of Ab, and remained in the bath throughout the experiment. fEPSP slopes were normalized to starting amplitude in each slice prior to averaging across slices, and all points are mean ±SEM of 7 slices in each group. Statistics were determined by Student's t-test for unpaired data.

### EQUIVALENTS

While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

Unless otherwise indicated, all numbers expressing quantities of ingredients, reaction conditions, parameters, descriptive features and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present invention.

All publications and patents mentioned herein, including those items listed below, are hereby incorporated by reference in their entirety as if each individual publication or patent was specifically and individually indicated to be incorporated by reference. In case of conflict, the present application, including any definitions herein, will control.

What is claimed is:

1. A method of treating a disorder associated with elevated NMDAR antibodies in a patient in need thereof, comprising administering to the patient a pharmaceutically effective amount of a spiro-β-lactam compound.

2. The method of claim 1, wherein the disorder associated with elevated NMDAR antibodies is anti-NMDAR encephalitis.

3. The method of claim 1 or 2, wherein the patient is also suffering from a germ-cell tumor.

4. The method of claim 3, wherein the tumor is an ovarian or testicular teratoma.

5. The method of any one of claims 1-4, wherein the patient is also suffering from cancer and/or an autoimmune disease.

6. The method of any one of claims 1-5, further comprising identifying the patient as having NMDAR IgA, IgM, and/or IgG isotype antibodies.

7. The method of any one of claims 1-6, further comprising identifying the patient as having NMDAR IgG isotype antibodies.

8. The method of any one of claims 1-7, wherein the disorder associated with elevated NMDAR antibodies is an immunotherapy-responsive dementia or psychiatric manifestation.

9. The method of claim 8, wherein the immunotherapy-responsive dementia is selected from the group consisting of unclassified dementia, progressive supranuclear palsy, corticobasal syndrome, frontotemporal dementia, Lewy body dementia, and primary progressive aphasia.

10. The method of any one of claims 1-7 wherein the patient suffers from progressive nonfluent aphasia.

11. The method of any one of claims 1-7, wherein the disorder associated with elevated NMDAR antibodies is an immunotherapy-responsive a neurodegenerative disorder without dementia.

12. The method of claim 11, wherein the neurodegenerative disorder without dementia is selected from the group consisting of motor neuron disease, Parkinson's disease without dementia, multiple system atrophy , spinocerebellar ataxia, and idiopathic sporadic ataxia.

13. The method of any one of claims 1-7 wherein the disorder associated with elevated NMDAR antibodies is an immunotherapy-responsive schizophrenia.

14. The method of any one of claims 1-13, wherein the disorder is Rasmussen's encephalitis.

15. The method of any one of claims 1-14, wherein the spiro-β-lactam compound is represented by formula (I) or (II): 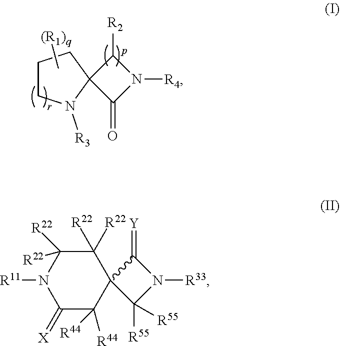
or a pharmaceutically acceptable salt, stereoisomer or N-oxide thereof.
wherein,
p is 1, 2, or 3;
q is 0, 1, 2 or 3;
r is 0, 1, 2, or 3;
R₁ is selected, for each occurrence, from the group consisting of hydrogen, halogen, cyano, hydroxyl, C₁₋₆alkyl, phenyl, —C(O)—C₁₋₆alkyl, and —C(O)—O—C₁₋₆alkyl;
R2 is selected for each occurrence from the group consisting of hydrogen, halogen, cyano, hydroxyl, C₁₋₆alkyl, and phenyl;
R₃ is selected from the group consisting of hydrogen, C₁₋₆alkyl, C(O)—C₁₋₆alkyl, S(O)_{w}—C₁₋₆alkyl (w is 0, 1 or 2), and C(O)—NH—C₁₋₆alkyl, wherein C₁₋₆alkyl is optionally substituted by one, two or three substituents each independently selected from the group consisting of OH, NR^{a}R^{b}, heteroaryl, phenyl, halogen, cyano, —C(O)—C₁₋₆alkyl, —C(O)—O—C₁₋₆alkyl, phenyl, and heteroaryl;
R₄ is selected from the group consisting of: an amino acid, C₁₋₆alkyl, wherein C₁₋₆alkyl is optionally substituted by one, two or three substituents each independently selected from the group consisting of OH, NR^{a}R^{b}, C(O)NR^{a}R^{b}, C(O)—C₁₋₆alkyl, C(O)—O—C₁₋₆alkyl, phenyl, heteroaryl, or heterocycle), phenyl, heteroaryl, S(O)_{w}—C₁₋₆alkyl (w is 0, 1 or 2); R^{a} and R^{b} are each independently for each occurrence selected from the group consisting of hydrogen, —C₁-C₄alkyl, and —CH₂-phenyl; or R^{a} and R^{b} taken together with the nitrogen to which they are attached form a 4-7 membered heterocyclic ring;
R¹¹ is selected from the group consisting of hydrogen, —C₁-C₆alkyl, —C(O)—C₁-C₆alkyl, —C(O)—O—C₁-C₆alkyl, —C₁-C₆alkylene-C₁-C₆cycloalkyl, and phenyl;
R²² is independently selected for each occurrence from the group consisting of hydrogen, cyano, —C₁-C₆alkyl, and halogen;
R³³ is selected from the group consisting of hydrogen, —C₁-C₆alkyl, —C(O)—R³¹, —C(O)—O—R³², and phenyl; wherein R³¹ is selected from the group consisting of hydrogen, —C₁-C₆alkyl, —C₁-C₆haloalkyl, —C₃-C₆cycloalkyl, and phenyl; R³² is selected from the group consisting of hydrogen, —C₁-C₆alkyl, —C₁-C₆haloalkyl, —C₃-C₆cycloalkyl, and phenyl; wherein any aforementioned C₁-C₆alkyl, independently for each occurrence, is optionally substituted by one, two or three substituents each independently selected from —C(O)NR^{a}R^{b}, —NR^{a}R^{b}, hydroxyl, —SH, phenyl, —O—CH₂-phenyl, and halogen; and any aforementioned phenyl, independently for each occurrence, is optionally substituted by one, two or three substituents each independently selected from —C(O)NR^{a}R^{b}, —NR^{a}R^{b}, —C₁-C₃alkoxy, hydroxyl, and halogen;
R⁴⁴ is independently selected for each occurrence from the group consisting of hydrogen, halogen, hydroxyl, cyano, phenyl, —C₁-C₄alkyl, —C₂₋₄alkenyl, —C₁₋₄alkoxy, —C(O)NR^{a}R^{b}, —NR^{a}R^{b}, —N(R^{a})-phenyl, —N(R^{a})—C₁-C₆alkylene-phenyl, —N(R^{a})—C(O)—C₁-C₆alkyl, —N(R^{a})—C(O)—C₁-C₆alkylene-phenyl, —N(R^{a})—C(O)—O—C₁-C₆alkyl, and —N(R^{a})—C(O)—O—C₁-C₆alkylene-phenyl; wherein C₁-C₄alkyl, C₁-C₆alkylene, C₂-C₄alkenyl, C₁-C₄alkoxy, and phenyl are optionally substituted by one or more substituents selected from R^{P}; or two R⁴⁴ moieties, when present on adjacent carbons, form a 3-membered carbocyclic ring taken together with the adjacent carbons to which they are attached, optionally substituted by one or two substituents independently selected from the group consisting of halogen, hydroxyl, —C₁-C₃alkyl, —C₁-C₃alkoxy, —C(O)NR^{a}R^{b}, and —NR^{a}R^{b}; R^{a} and R^{b} are each independently for each occurrence selected from the group consisting of hydrogen, —C₁-C₄alkyl, and —CH₂-phenyl; or R^{a} and R^{b} taken together with the nitrogen to which they are attached form a 4-7 membered heterocyclic ring;
R⁵⁵ is independently selected for each occurrence from the group consisting of hydrogen, —C₁-C₃alkyl, phenyl, and halogen; wherein phenyl is optionally substituted by one or more substituents selected from R^{P}; or two R⁵⁵ moieties together with the carbon to which they are attached form a carbonyl moiety or thiocarbonyl moiety.

16. The method of claim 15, wherein the compound is represented by Formula (I): 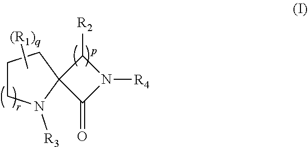
or a pharmaceutically acceptable salt, stereoisomer or N-oxide thereof.
wherein,
p is 1, 2, or 3;
q is 0, 1, 2 or 3;
r is 0, 1, 2, or 3.

17. The method of claim 15 or 16, wherein R₁ is H.

18. The method of any one of claims 15-17, wherein R₂ is H.

19. The method of any one of claims 15-18, wherein R₃ is selected from a group consisting of hydrogen, C₁₋₆alkyl , C(O)—C₁₋₆alkyl, and S(O)_{w}—C₁₋₆alkyl (w is 0, 1 or 2) .

20. The method of any one of claims 15-19, wherein R₃ is hydrogen or C(O)—C₁₋₆alkyl;
wherein C₁₋₆alkyl is selected from a group consisting of methyl, ethyl, and isopropyl.

21. The method of any one of claims 15-20, wherein R₃ is:

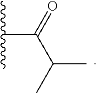 [SMILES: *C(=O)C(C)C]

22. The method of any one of claims 15-21, wherein R₄ is an amino acid and C₁₋₆alkyl; wherein C₁₋₆alkyl is optionally substituted by one, two or three substituents each independently selected from the group consisting of OH, NR^{a}R^{b}, —C(O)NR^{a}R^{b}, C(O)—C₁₋₆alkyl, —C(O)—O—C₁₋₆alkyl, phenyl, heteroaryl, and heterocycle; wherein, R^{a} and R^{b} are each independently selected for each occurrence from the group consisting of hydrogen and —C₁-C₆alkyl.

23. The method of any one of claims 15-22, wherein R₄ is: 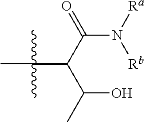
wherein, R^{a} and R^{b} are each independently selected for each occurrence from the group consisting of hydrogen and —C₁-C₆alkyl.

24. The method of any one of claims 15-23, wherein R₄ is:

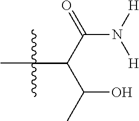 [SMILES: *C(C(N)=O)C(C)O]

25. The method of any one of claims 15-24, wherein the compound is 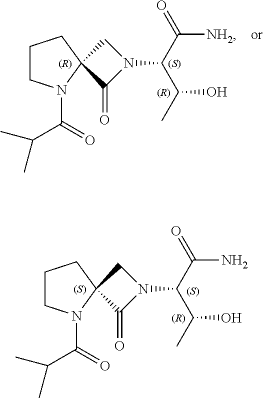
or a pharmaceutically acceptable salt, stereoisomer or N-oxide thereof.

26. The method of any one of claims 1-14, wherein the spiro-β-lactam compound is represented by Formula (II): 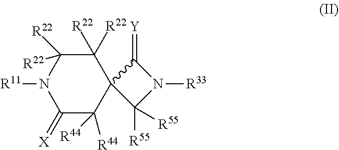
or a pharmaceutically acceptable salt, stereoisomer or N-oxide thereof.

27. The method of claim 26, wherein R¹¹ is hydrogen, and —C₁-C₆alkyl, wherein —C₁-C₆alkyl is optionally substituted by phenyl, where phenyl is optionally substituted by one, two or three substituents each independently selected from —C₁-C₃alkoxy and fluoro.

28. The method of any one of claims 26-27, wherein R¹¹ is hydrogen.

29. The method of any one of claims 26-28, wherein R²² is independently selected for each occurrence from the group consisting of hydrogen, and —C₁-C₆alkyl.

30. The method of any one of claims 26-29, wherein R²² is hydrogen.

31. The method of any one of claims 26-30, wherein R⁴⁴ is independently selected for each occurrence from the group consisting of hydrogen, halogen, hydroxyl, cyano, phenyl, —C₁-C₄alkyl, —C₂₋₄alkenyl, —C₁₋₄alkoxy, —C(O)NR^{a}R^{b}, —NR^{a}R^{b}; where R^{a} and R^{b} are each independently for each occurrence selected from the group consisting of hydrogen, —C₁-C₄alkyl, and —CH₂-phenyl.

32. The method of any one of claims 26-31, wherein R⁴⁴ is hydrogen.

33. The method of any one of claims 26-32, wherein R⁵⁵ is independently selected for each occurrence from the group consisting of hydrogen, —C₁-C₃alkyl, and halogen

34. The method of any one of claims 26-33, wherein R⁵⁵ is hydrogen.

35. The method of any one of claims 26-34, wherein R³³ is: 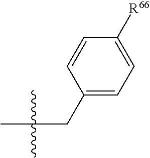
where:
R⁶⁶ is selected from the group consisting of hydrogen, halogen, —C₁-C₃alkoxy.

36. The method of any one of claims 26-35, wherein R⁶⁶ is methoxy.

37. The method of any one of claims 26-36, wherein the compound is: 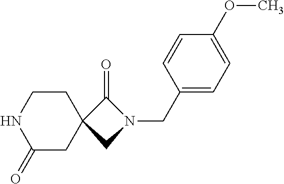
or a pharmaceutically acceptable salt, stereoisomer or N-oxide thereof.
